(19) United States
(12) Patent Application Publication
(10) Pub. No.: US 2026/0224588 A1
(43) Pub. Date: Aug. 6, 2026

(54) BENZODIAZEPINONE-DERIVED INHIBITORS OF EGFR AND HER2

(71) Applicants: The Research Foundation for the State University of New York, Amherst, NY (US); Eberhard Karls Universität Tübingen, Tübingen (DE)

(72) Inventors: David Heppner, Snyder, NY (US); Stefan A. Laufer, Tübingen (DE); Florian Wittlinger, Tübingen (DE); Blessing Ogboo, Buffalo, NY (US); Calvin Pham, Buffalo, NY (US); Alexander Rasch, Tübingen (DE)

(21) Appl. No.: 19/154,885

(22) PCT Filed: Feb. 8, 2024
(86) PCT No.: PCT/US2024/015004

§ 371 Date: Aug. 8, 2025

Related U.S. Application Data
(60) Provisional application No. 63/483,871, filed on Feb. 8, 2023.

(51) Int. Cl. A61K 31/5513; A61P 35/00; C07D 401/14

(52) U.S. Cl. CPC A61K 31/5513; A61P 35/00; C07D 401/14

(57) ABSTRACT

Provided are compounds having the following structure: (I) wherein R′ is a halogen, a substituted aliphatic group (e.g., substituted alkyl group), an unsubstituted aliphatic group (e.g., unsubstituted aliphatic group), or absent; R² is a halogen, a substituted aliphatic group (e.g., substituted alky 1 group), an unsubstituted aliphatic group (e.g., unsubstituted aliphatic group), or absent; R² is a substituted aliphatic group (e.g., substituted alkyl group), unsubstituted aliphatic group (e.g., unsubstituted alkyl group), a thioether group, or absent; R⁴ is an amide or a secondary amine; and L is a linker. Also provided are compositions of the compounds and methods of using the compounds. The compounds may be used to treat individuals having or suspected of having cancer.

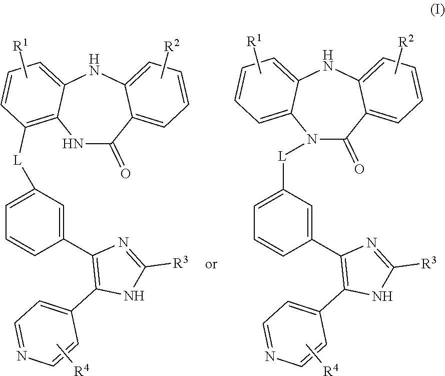

### CROSS REFERENCE TO RELATED APPLICATION

This application claims the benefit of U.S. Provisional Patent Application No. 63/483,871, filed Feb. 8, 2023, the disclosure of which is incorporated herein by reference.

### STATEMENT REGARDING FEDERALLY SPONSORED RESEARCH

This invention was made with government support under grant number TR001412 awarded by National Institutes of Health. The government has certain rights in the invention.

### BACKGROUND OF THE DISCLOSURE

Fragment-based drug discovery (FBDD) comprises a variety of strategies to initiate the development of small-molecule drugs, and usually utilizes hits acquired from a biophysical compound library screening, with low affinity in target binding, low structural complexity and low molecular weight, which are modified to drug-like compounds via molecular enlargement. Generally, fragments bound at adjacent sites within a promising target protein inform the rational design of linker structures that bridge these pockets. The desired outcome is for the resulting molecule to bind far stronger compared to the original fragments producing a highly efficacious starting point for lead discovery. Linking of fragments continues to be the major bottleneck and origin of failure in this technique. Based on theoretical considerations, or a handful of successful cases. Despite previous studies having offered design strategies for FBDD linkers, finding an ideal linker is still considered a major challenge.

A model system to study FBDD connections is the established kinase drug target the epidermal growth factor receptor (EGFR). The subject of decades of drug development, tumors harboring the EGFR activating mutations L858R (LR) and exon19del, as well as drug resistant T790M (TM) gatekeeper and C797S (CS) mutants, have led to a diverse set of small-molecule inhibitors. A growing number of mutant-selective ATP-site (orthosteric) inhibitors are known in addition to the more distinct allosteric (type 3) inhibitors. Structurally, the allosteric pocket is located adjacent to the ATP site allowing for co-binding of inhibitors to both sites, which are associated with positive cooperativity potentially and synergy in vivo. Additionally, there are recently emerged examples of molecules that are designed to bind simultaneously to the ATP and allosteric sites in EGFR.

### SUMMARY OF THE DISCLOSURE

Fragment-based drug design (FBDD) is a well-accepted strategy in drug development but hindered by challenges in selecting proper fragment linker structures. In an aspect, the present disclosure provides bivalent EGFR inhibitors that span the ATP and allosteric pockets as a model system for understanding the molecular factors that drive potency based on fragment linker structure. Structurally characterized compounds exhibiting high and low biochemical IC₅₀ values experimentally confirm the importance of designing linkers for proper fragment binding and enabling additional intermolecular interactions. The resulting compounds, such as, for example, compound 4, are active in human cancer cells, selective, and metabolically stable, offering a distinctive example of how structure-guided linker optimization can afford active leads in FBDD.

Due to the proximity of the drug-binding allosteric pocket and drug-binding ATP site, the disclosure provides fragment-based linking of compounds that bind to the ATP and allosteric EGFR inhibitors. ATP-competitive inhibitors were selected based on structurally characterized trisubstituted imidazole molecules, and the EGFR 5,10-dihydro-11H-dibenzo[b,e][1,4]diazepin-11-one (benzo) allosteric inhibitor DDC4002 (FIG. 11, FIG. 4). A set of bivalent ATP-allosteric inhibitors were synthesized, bridged by an N-linked methylene (1) and C-linked amide (2-4) (FIG. 11). A Suzuki-Miyaura cross-coupling reaction-based strategy was used for the combination of the fragments of N-linked derivative 1 (Scheme 1), while fragments of the C-linked derivatives were assembled modular before linking them via mild amide coupling conditions (Schemes 3-6).

In an aspect, the present disclosure provides compounds having the following structure:

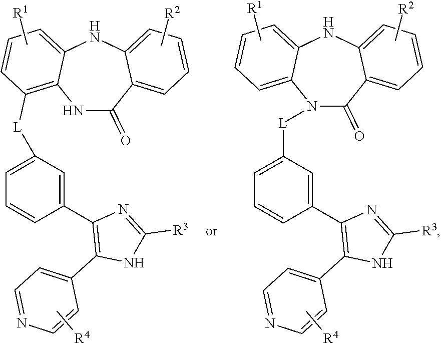

wherein R¹ is a halogen, a substituted aliphatic group (e.g., substituted alkyl group), an unsubstituted aliphatic group (e.g., unsubstituted aliphatic group), or absent; R² is a halogen, a substituted aliphatic group (e.g., substituted alkyl group), an unsubstituted aliphatic group (e.g., unsubstituted aliphatic group), or absent; R³ is a substituted aliphatic group (e.g., substituted alkyl group), unsubstituted aliphatic group (e.g., unsubstituted alkyl group), a thioether group, or absent; R⁴ is an amide or a secondary amine; and L is a linker. When a group is absent, it means the substituent has been replaced with an —H.

In another aspect, the present disclosure provides a composition comprising the compound of the disclosure.

In another aspect, the present disclosure provides a method of treating an individual having or suspected of having cancer, comprising administering a therapeutically effective amount of the compound or a composition comprising the compound to the individual.

In another aspect of the present disclosure, a kit is provided, comprising a composition comprising the compound of the disclosure, or constituents to prepare a composition comprising the compound of the disclosure.

### BRIEF DESCRIPTION OF THE FIGURES

For a fuller understanding of the nature and objects of the disclosure, reference should be made to the following detailed description taken in conjunction with the accompanying figures.

FIG. 1. Binding modes of A) 1 (PDB ID 8FV3) and B) 2 (PDB ID 8FV4) in complex with EGFR(T790M/V948R). C) Top view overlay of 1 and 2 cocrystal structures from a showing “inward versus outward” pucker benzo conformations. D) Overlay of 2 and DDC4002 (6P1D).

FIG. 2. A) The covalent bivalent EGFR inhibitor 4 reduces EGFR (L858R/T790M), ERK1/2, and AKT phosphorylation in H1975 lung cancer cells. B) The reversible binding 2 suppresses EGFR phosphorylation in H1975 cells. C) 4 effectively ablates EGFR (delE746-A750) phosphorylation in HCC827 cells. All experiments performed after 6 hours treatments of inhibitors or DMSO controls and Western blots are representative of at least three independent experiments.

FIG. 3. Overlay of LN2725 (PDB ID 6V5P) and DDC4002 (PDB ID 6P1D).

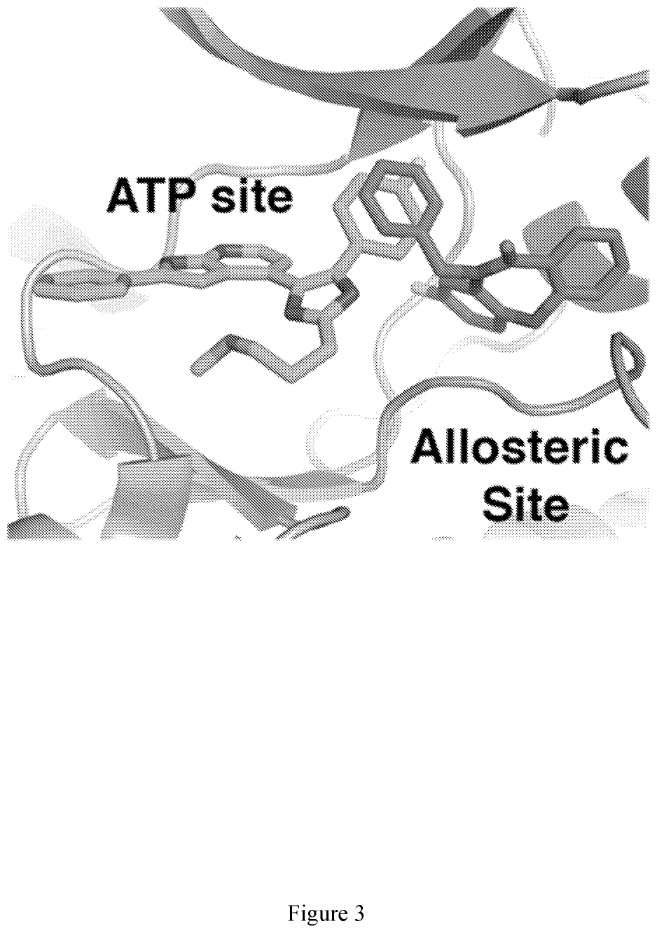

FIG. 4. Crystal structure densities of 1 (PDB ID 8FV3) and 2 (PDB ID 8FV4) in complex with EGFR(T790M/V948R). A) Chemical structure of 1. B) 2Fo-Fc electron density map of 1 and surrounding side chains and solvent waters. C) Fo-Fc simulated annealing omit map of 1. Electron density maps for cocrystal structures of 1 and 2 in complex with EGFR(T790M/V948R). D) Chemical structure of 2. E) 2Fo-Fc electron density map of 2 and surrounding side chains and solvent waters. F) Fo-Fc simulated annealing omit map of 1.

FIG. 5. HTRF dose-dependent curves for reversible binding compounds 1, 2, 3.

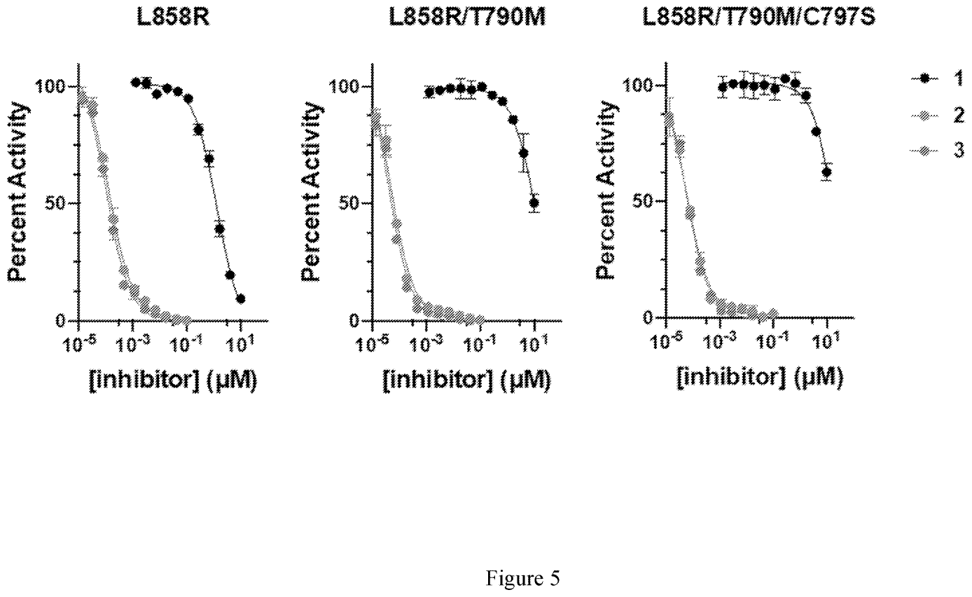

FIG. 6. Schematic showcasing the lack of rotational flexibility in (A) 1 and how the linker in (B) 2-4 enables free rotation for the allosteric group.

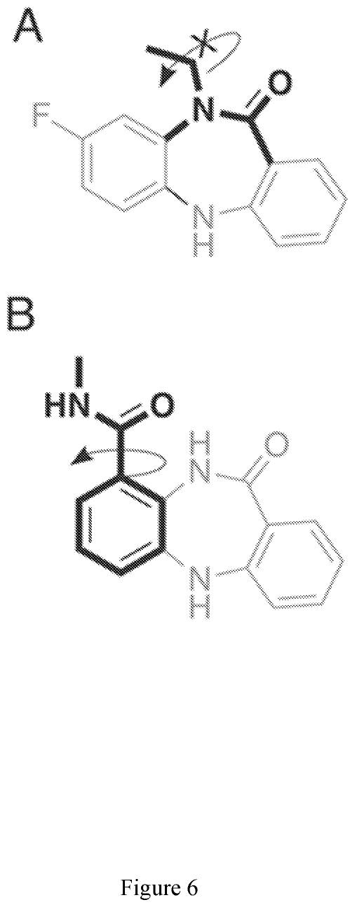

FIG. 7. Selected comparisons of the geometries in A) 1 and B) 2 from co-crystal structures, which show similar distances from the imidazole at the ATP site and the benzo phenyl ring anchored in the hydrophobic pocket. C) The angle between the benzo phenyl rings in the 2 “inward” and 1 “outward” pucker and ring-to-ring distance.

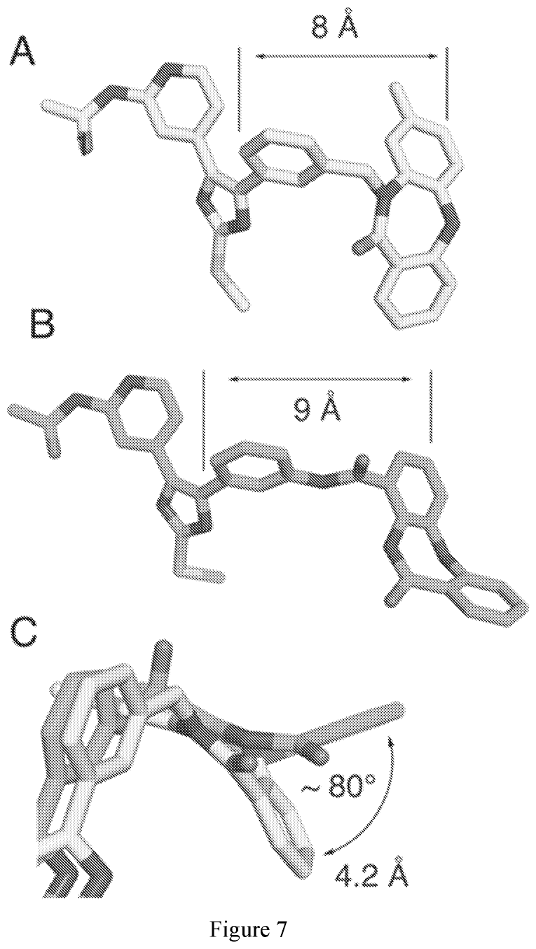

FIG. 8. Impact to phosphorylated EGFR upon 6-hour dosing of 1 and 3 in H1975 cells. Representative of N=3 independent experiments.

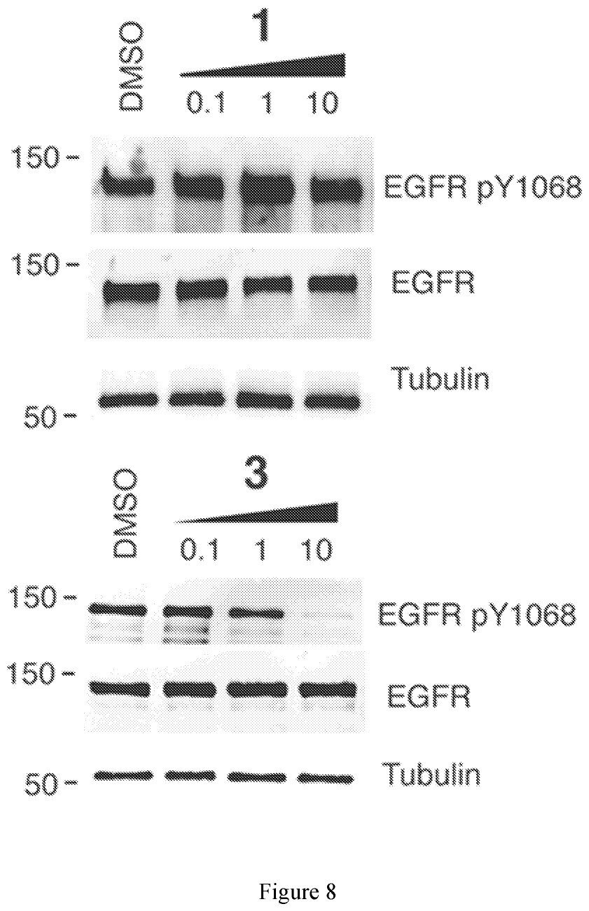

FIG. 9. A) Compound 4 was docked into the EGFR(T790M/V948R) kinase domain created from the co-crystal structure with 2 using Schrodinger Glide software (Glide Score=−18.29, Docking=−16.96). The docking pose shows the expected binding pose and positioning of the acrylamide warhead in proximity to the C797 residue. B) Superposition of the docking pose of compound 4 (red) and EGFR(T790M/V948R) co-crystal structures of the bivalent 2 and ATP-site covalent inhibitor 7 (PDB ID 6V6K) indicating that 4 fully spans the orthosteric and allosteric pocket.

FIG. 10. Metabolic stability determination of 4 in human liver microsomes, compared to a negative control containing bovine serum albumin instead of liver microsomes and a positive control using verapamil instead of compound.

FIG. 11. Chemical structures of bivalent ATP-allosteric inhibitors consisting of N-linked reversible (1) as well as C-linked reversible (2,3,5) and covalent (4) scaffolds. Parent ATP site imidazole inhibitors (LN2057) as well as dibenzodiazepinone allosteric inhibitors (DDC4002 and EAI002).

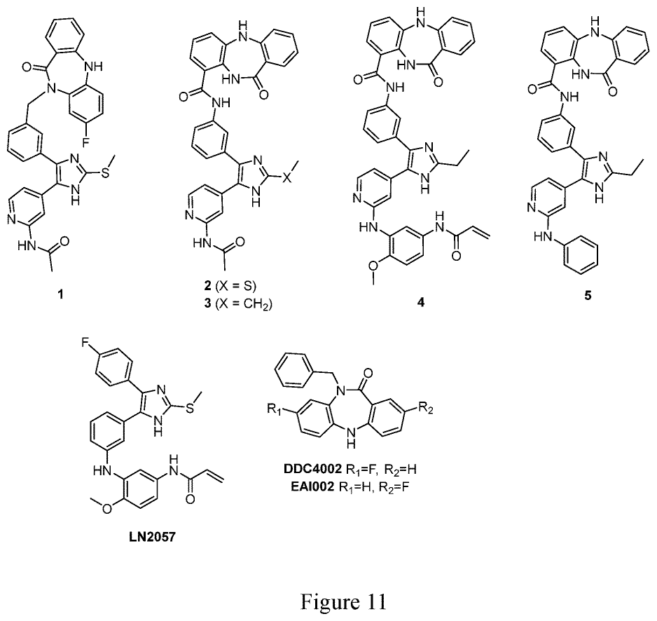

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

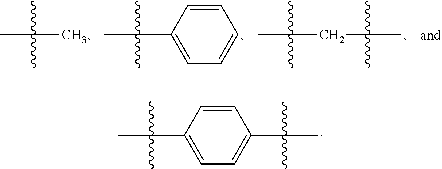

As used herein, unless otherwise indicated, the term “alkyl” or “alkyl group” refers to branched or unbranched, linear saturated hydrocarbon groups and/or cyclic hydrocarbon groups. Examples of alkyl groups include, but are not limited to, methyl groups, ethyl groups, propyl groups, butyl groups, isopropyl groups, tert-butyl groups, cyclopropyl groups, cyclopentyl groups, cyclohexyl groups, and the like. Alkyl groups are saturated groups, unless it is a cyclic group. For example, an alkyl group is a C₁ to C₂₀ alkyl group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀). The alkyl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof.

As used herein, the term “cycloalkyl” or “cycloalkyl group” refers to a cyclic hydrocarbon group, e.g., cyclopropyl, cyclobutyl, cyclohexyl, and cyclopentyl groups. Cycloalkyl groups can be saturated or partially unsaturated ring systems optionally substituted with, for example, one to three substituents. Each substituent is independently chosen from alkyl, —NH₂, oxo (═O), phenyl, haloalkyl (e.g., —CF₃), halo (e.g., —F, —Cl, —Br, —I), alkoxy, and —OH groups. Additionally, alkyl substituents may be substituted with various other functional groups. Additional non-limiting examples include aliphatic groups (e.g., alkyl groups, alkenyl groups, alkynyl groups, and the like), halogenated aliphatic groups (e.g., trifluoromethyl group), aryl groups, halogenated aryl groups, alkoxide groups, nitro groups, carboxylate groups, carboxylic acids, ether groups, alkyne groups (e.g., acetylenyl groups and the like), and the like, and combinations thereof.

As used herein, unless otherwise indicated, the term “aryl” or “aryl group” refers to C₅ to C₁₆ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, or C₁₆). An aryl group may also be referred to as an aromatic group. The aryl groups may comprise polyaryl groups such as, for example, fused rings, biaryl groups, or a combination thereof. The aryl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), fused ring groups (e.g., naphthyl groups and the like), hydroxy benzyl groups, tolyl groups, xylyl groups, and the like.

As used herein, the term “heteroaryl” or “hereteroaryl” refers to a monocyclic or bicyclic ring system comprising one or two aromatic rings and containing at least one nitrogen or oxygen atom in an aromatic ring. Unless otherwise indicated, a heteroaryl group can be unsubstituted or substituted with one or more, and in particular one or two, substituents. Non-limiting examples of substituents include halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof. Examples of heteroaryl groups include, benzofuranyl, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl groups, and substituents analogs of any of the foregoing heteroaryl groups.

As used herein, unless otherwise indicated, the term “alkoxy” or “alkoxy group” refers to

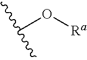

where R^{a} is a linear, branched or cyclic C₁-C₆ alkyl group, including all integer numbers of carbons and ranges of numbers of carbons therebetween. For example, suitable alkoxy groups include methoxy, ethoxy, propoxy, iso-propoxy, butoxy, sec-butoxy, tert-butoxy, and hexoxy groups. Additionally, alkyl substituents can be substituted with various other functional groups, e.g. functional groups disclosed herein.

As used herein, unless otherwise indicated, the term “amino” or “amino group” refers to

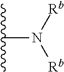

where each R^{b} is selected independently from the group consisting of hydrogen atom, substituted or unsubstituted C₁-C₁₀ alkyl, including all integer numbers of carbons and ranges of numbers of carbons therebetween, substituted or unsubstituted phenyl, substituted or unsubstituted heteroaryl, substituted carbonyl, substituted sulfonyl, haloalkyl, and substituted or unsubstituted benzyl groups. Amino groups may be referred to as “amine groups” or “amines.” The amine groups may further be described by their level of functionalization (e.g., primary amine, secondary amine, tertiary amine, or quaternary amine).

As used herein, unless otherwise indicated, the term “benzyl” or “benzyl group” refers to

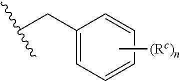

where R^{c} is a substituent on the phenyl ring and n is from 0 to 5. The substituents can be the same or different. For example, the substituents on the benzyl group include substituted or unsubstituted alkyl, —NH₂, phenyl, haloalkyl (e.g., —CF₃), halo (e.g., —F, —Cl, —Br, —I), alkoxy (e.g., —OMe), and —OH groups.

As used herein, unless otherwise indicated, halogen means fluorine, chlorine, bromine, and iodine, and halo means fluoro, chloro, bromo, and iodo.

As used herein, unless otherwise indicated, the term “phenoxy” or “phenoxy group” (—OPh) refers to

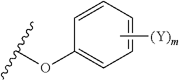

where each Y is independently selected from the group consisting of F, Cl, Br, and I and m can be 0, 1 or 2.

As used herein, unless otherwise indicated, the term “phenyl” or “phenyl group” means

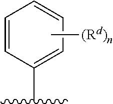

where each R^{d} is an independent substituent on the phenyl group and n is from 0 to 5. The substituents at different occurrences can be the same or different. For example, the substituents on the phenyl group include substituted or unsubstituted C₁-C₆ alkyl, including all integer numbers of carbons and ranges of numbers of carbons therebetween, substituted or unsubstituted amino, haloalkyl (e.g., —CF₃), halo (e.g., —F, —Cl, —Br, —I), substituted or unsubstituted alkoxy (e.g., —OMe), and sulfonyl group. In certain instances, two adjacent R groups can be connected through to form a dioxolyl group.

In an aspect, the present disclosure provides compounds having the following structure:

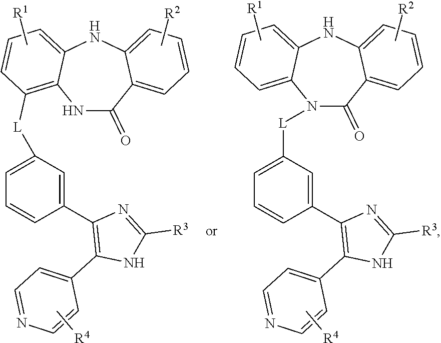

wherein R¹ is a halogen, a substituted aliphatic group (e.g., substituted alkyl group), an unsubstituted aliphatic group (e.g., unsubstituted aliphatic group), or absent; R² is a halogen, a substituted aliphatic group (e.g., substituted alkyl group), an unsubstituted aliphatic group (e.g., unsubstituted aliphatic group), or absent; R³ is a substituted aliphatic group (e.g., substituted alkyl group), unsubstituted aliphatic group (e.g., unsubstituted alkyl group), a thioether group, or absent; R⁴ is an amide or a secondary amine; and L is a linker. When a group is absent, it means the substituent has been replaced with an —H.

A compound of the present disclosure may have various R¹ groups. In various embodiments, R¹ is a halogen, such as, for example, —I, —F, —Br, or —C₁. In various other embodiments, R¹ is an aliphatic group (e.g., a substituted aliphatic group or unsubstituted aliphatic groups. The aliphatic groups may be linear or branched aliphatic groups, which may be substituted or unsubstituted. In various embodiments, the aliphatic groups, which may be substituted or unsubstituted and/or linear or branched, the aliphatic groups are alkyl groups. In various embodiments, R¹ is absent and replaced with —H.

A compound of the present disclosure may have various R² groups. In various embodiments, R² is a halogen, such as, for example, —I, —F, —Br, or —Cl. In various other embodiments, R² is an aliphatic group (e.g., a substituted aliphatic group or unsubstituted aliphatic groups. The aliphatic groups may be linear or branched aliphatic groups, which may be substituted or unsubstituted. In various embodiments, the aliphatic groups, which may be substituted or unsubstituted and/or linear or branched, the aliphatic groups are alkyl groups. In various embodiments, R² is absent and replaced with —H.

A compound of the present disclosure may have various R³ groups. In various other embodiments, R³ is an aliphatic group (e.g., a substituted aliphatic group or unsubstituted aliphatic groups. The aliphatic groups may be linear or branched aliphatic groups, which may be substituted or unsubstituted. In various embodiments, the aliphatic groups, which may be substituted or unsubstituted and/or linear or branched, the aliphatic groups are alkyl groups. Examples of alkyl groups include, but are not limited to:

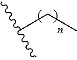

and the like,
where n is 0, 1, 2, or 3. In various other examples, R³ is a thioether. Examples of thioethers include, but are not limited to:

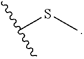 [SMILES: *SC]

and the like.

A compound of the present disclosure may have various R⁴ groups. In various embodiments, R⁴ is an amide or a secondary amine. For example, the amide may be an alkyl amide. Examples of amides include but are not limited to:

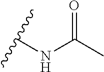 [SMILES: *NC(C)=O]

and the like.
In various examples, the secondary amine is a aryl amine, where the nitrogen is bonded to an aryl group. The aryl group may be substituted or unsubstituted. For example, a substituted aryl group may have one or more substitutents. Examples of aryl substitutents are provided herein. Examples of secondary amine group include, but are not included to:

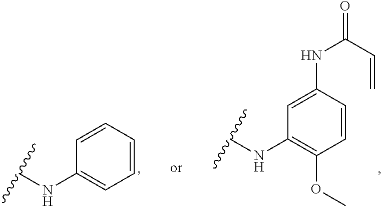 [SMILES: *Nc1ccccc1 ; *Nc1cc(NC(=O)C=C)ccc1OC]

and the like.

A compound of the present disclosure may have various L groups. L groups are bivalent linker groups, which may be referred to as linkers, linking groups, linker groups, L groups, or L. Examples of L groups included, but are not limited to, amides, secondary amines, ethers, diamides, sulfonamides, and aliphatic groups (e.g., alkyl groups). For example, amides, secondary amines, ethers, diamides, and sulfonamides may further comprise aliphatic groups. As an illustrative example, an amide comprising an aliphatic group may have the following structure:

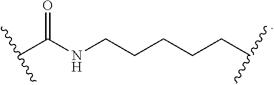 [SMILES: *CCCCCNC(*)=O]

Non-limiting examples of L groups include,

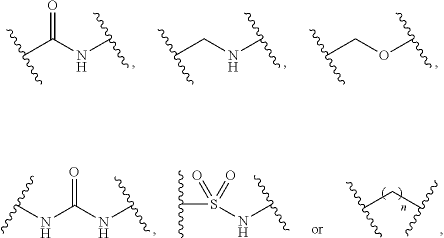

where n is 1, 2, or 3.

In various embodiments, a compound of the present disclosure has the following structure:

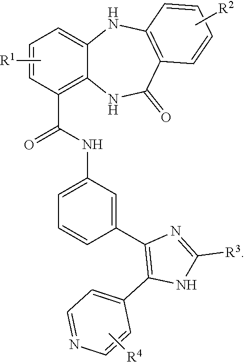

In embodiments, a compound of the present disclosure has the following structure:

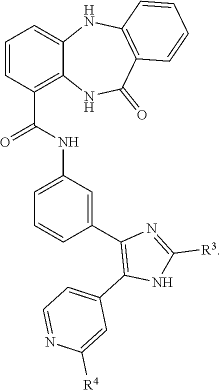

In embodiments, a compound of the present disclosure has the following structure:

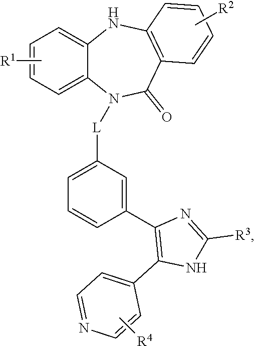

where L is

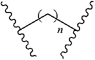

and n is 1, 2, or 3.

For example, a compound of the present disclosure has the following structure:

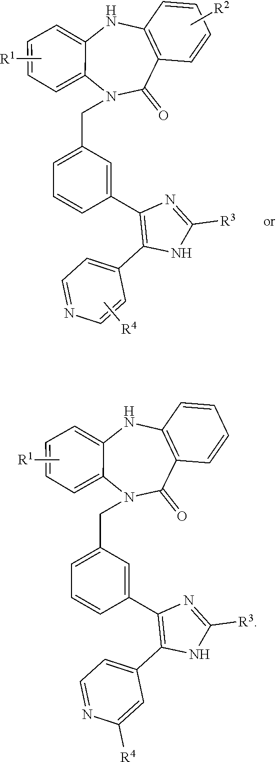

In various embodiments, a compound may have the following structure:

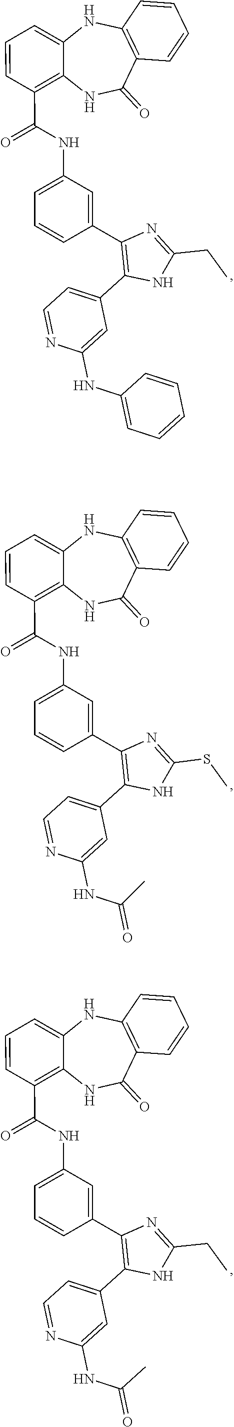 [SMILES: CCc1nc(-c2cccc(NC(=O)c3cccc4c3NC(=O)c3ccccc3N4)c2)c(-c2ccnc(Nc3ccccc3)c2)[nH]1 ; CSc1nc(-c2cccc(NC(=O)c3cccc4c3NC(=O)c3ccccc3N4)c2)c(-c2ccnc(NC(C)=O)c2)[nH]1 ; CCc1nc(-c2cccc(NC(=O)c3cccc4c3NC(=O)c3ccccc3N4)c2)c(-c2ccnc(NC(C)=O)c2)[nH]1]

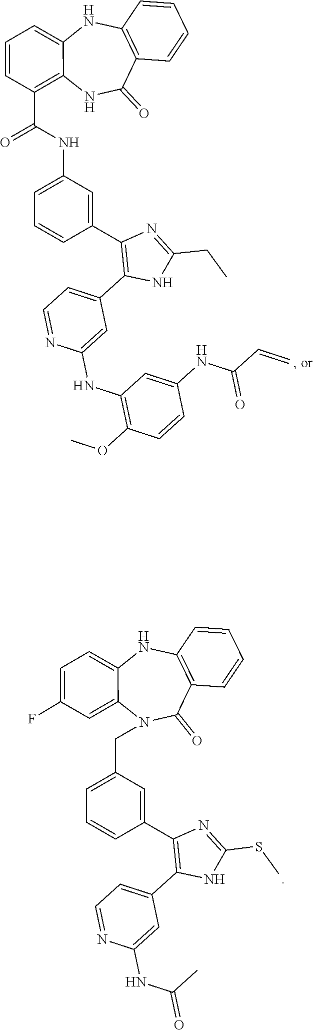 [SMILES: C=CC(=O)Nc1ccc(OC)c(Nc2cc(-c3[nH]c(CC)nc3-c3cccc(NC(=O)c4cccc5c4NC(=O)c4ccccc4N5)c3)ccn2)c1 ; CSc1nc(-c2cccc(CN3C(=O)c4ccccc4Nc4ccc(F)cc43)c2)c(-c2ccnc(NC(C)=O)c2)[nH]1]

In various embodiments, the compound may have the following structures:

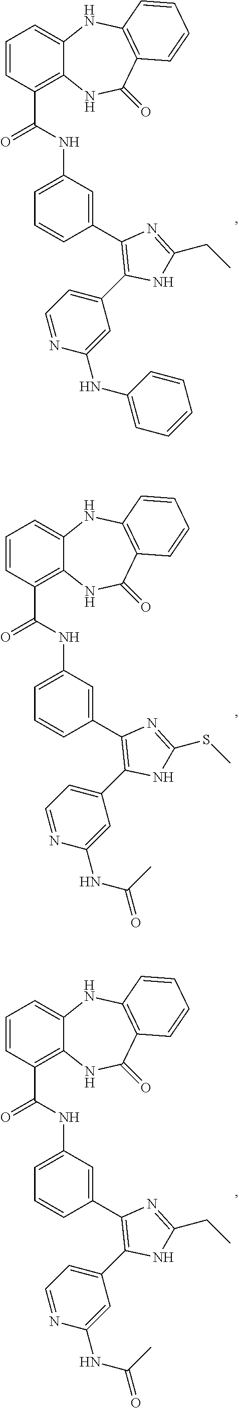 [SMILES: CCc1nc(-c2cccc(NC(=O)c3cccc4c3NC(=O)c3ccccc3N4)c2)c(-c2ccnc(Nc3ccccc3)c2)[nH]1 ; CSc1nc(-c2cccc(NC(=O)c3cccc4c3NC(=O)c3ccccc3N4)c2)c(-c2ccnc(NC(C)=O)c2)[nH]1 ; CCc1nc(-c2cccc(NC(=O)c3cccc4c3NC(=O)c3ccccc3N4)c2)c(-c2ccnc(NC(C)=O)c2)[nH]1]

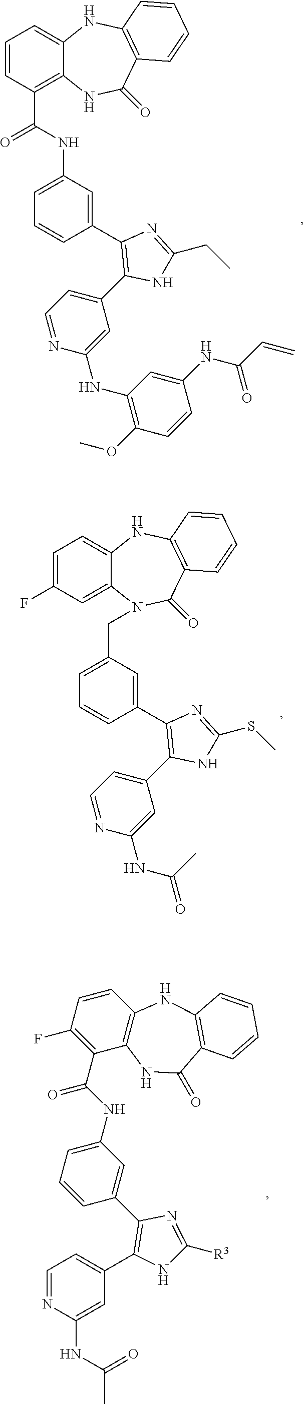

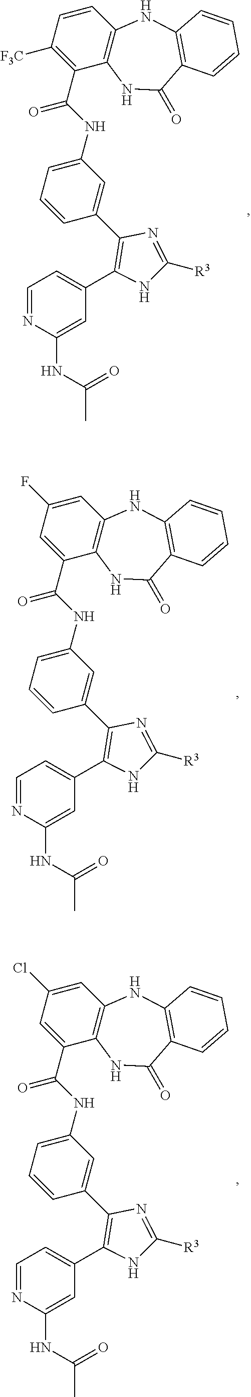

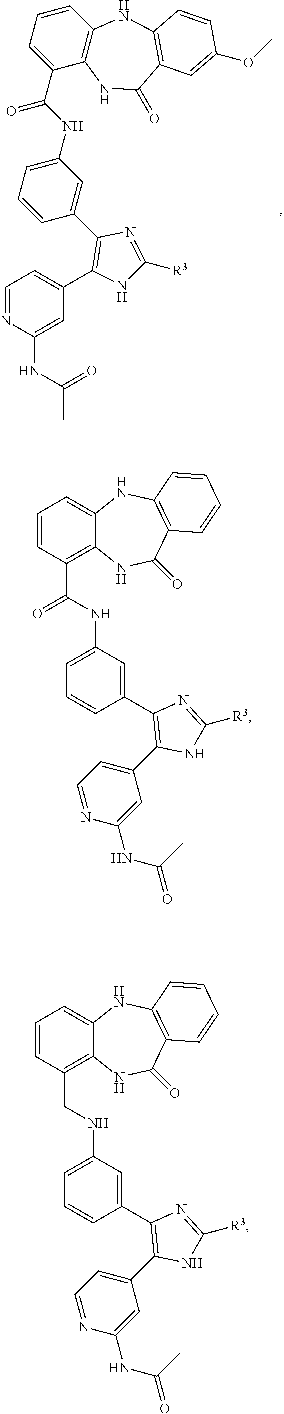

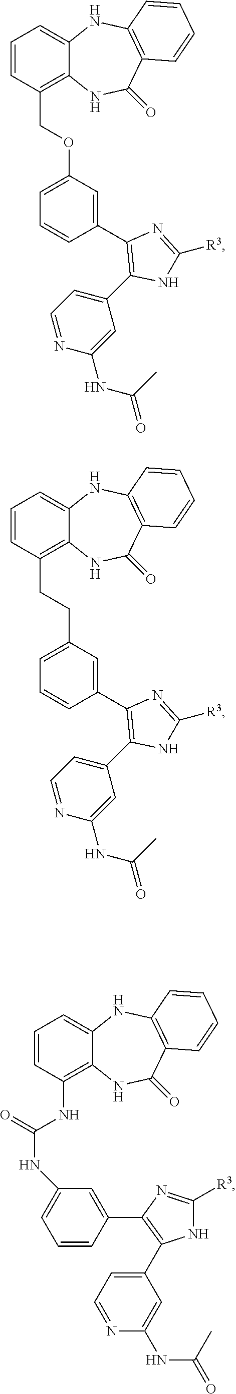

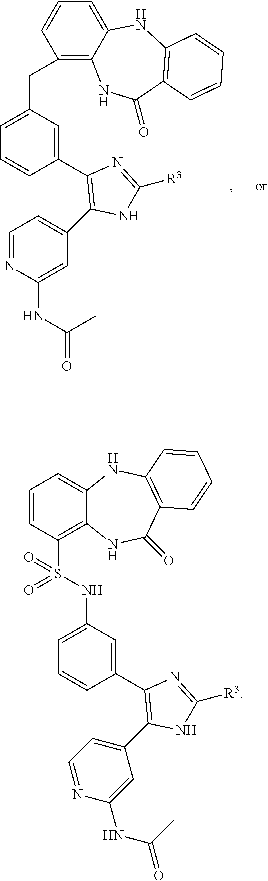

Prodrugs of a compound of the present disclosure also can be used as the compound in a method of the present disclosure. It is well established that a prodrug approach, wherein a compound is derivatized into a form suitable for formulation and/or administration, then released as a drug in vivo, has been successfully employed to transiently (e.g., bioreversibly) alter the physicochemical properties of the compound (see, H. Bundgaard, Ed., “Design of Prodrugs,” Elsevier, Amsterdam, (1985); R. B. Silverman, “The Organic Chemistry of Drug Design and Drug Action,” Academic Press, San Diego, chapter 8, (1992); K. M. Hillgren et al., Med. Res. Rev., 15, 83 (1995)).

Compounds of the present disclosure can contain one or more functional groups. The functional groups, if desired or necessary, can be modified to provide a prodrug. Suitable prodrugs include, for example, acid derivatives, such as amides and esters. It also is appreciated by those skilled in the art that N-oxides can be used as a prodrug.

The present disclosure includes all possible stereoisomers and geometric isomers of a compound of the present disclosure. The present disclosure includes both racemic compounds and optically active isomers. When a compound of the present disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8 (6), pages 883-888 (1997). Resolution of the final product, an intermediate, or a starting material can be achieved by any suitable method known in the art. Additionally, in situations where tautomers of a compound of the present disclosure are possible, the present disclosure is intended to include all tautomeric forms of the compounds.

Compounds of the disclosure may exist as salts. Pharmaceutically acceptable salts of the compounds of the disclosure generally are preferred in the methods of the disclosure. As used herein, the term “pharmaceutically acceptable salts” refers to salts or zwitterionic forms of a compound of the present disclosure. Salts of compounds of the present disclosure can be prepared during the final isolation and purification of the compounds or separately by reacting the compound with an acid having a suitable cation. The pharmaceutically acceptable salts of a compound of the present disclosure are acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds of the present disclosure appearing herein is intended to include a compound of the present disclosure as well as pharmaceutically acceptable salts, hydrates, or prodrugs thereof.

The compounds may exhibit wide variability in pharmacokinetic and physicochemical properties while still retaining desirable biological activity as described herein. For example solubility, e.g., log P, is variable while still retaining desirable biological activity as described herein.

In another aspect, the present disclosure provides a composition comprising the compound of the disclosure.

In various embodiments, the composition may further comprise a pharmaceutically acceptable carrier. Non-limiting examples of compositions include solutions, suspensions, emulsions, solid injectable compositions that are dissolved or suspended in a solvent before use, and the like. Injections may be prepared by dissolving, suspending, or emulsifying one or more of the active ingredient(s) in a diluent. Non-limiting examples of diluents include distilled water (e.g., for injection), physiological saline, vegetable oil, alcohol, and the like, and combinations thereof. Injections may contain, for example, stabilizers, solubilizers, suspending agents, emulsifiers, soothing agents, buffers, preservatives, and the like, and combinations thereof. Injections may be sterilized in the final formulation step or prepared by sterile procedure. A pharmaceutical composition of the disclosure may also be formulated into a sterile solid preparation, for example, by freeze-drying, and may be used after sterilized or dissolved in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, such as, for example, sodium carboxymethyl cellulose, ethyl cellulose, and cellulose acetate; powdered tragacanth; malt; gelatin; talc; excipients, such as, for example, cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example, glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; other non-toxic compatible substances employed in pharmaceutical formulations, and the like, and combinations thereof. Non-limiting examples of pharmaceutically acceptable carriers are found in: Remington: The Science and Practice of Pharmacy (2012) 22nd Edition, Philadelphia, PA. Lippincott Williams & Wilkins.

Compositions of the disclosure can comprise more than one pharmaceutical agent. For example, a first composition comprising a compound of the disclosure and a first pharmaceutical agent can be separately prepared from a composition which comprises the same compound of the disclosure and a second pharmaceutical agent, and such preparations can be mixed to provide a two-pronged (or more) approach to achieving the desired prophylaxis or therapy in an individual. Further, compositions of the disclosure can be prepared using mixed preparations of any of the compounds disclosed herein.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of a compound of the present disclosure include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to a compound of the disclosure, the composition may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The composition may be for administration to an individual in need of treatment.

In another aspect, the present disclosure provides a method of treating an individual having or suspected of having cancer, comprising administering a therapeutically effective amount of the compound or a composition comprising the compound to the individual.

In various embodiments, the individual is a human or non-human animal.

In embodiments, the composition may inhibit a mutant epidermal growth factor receptor (EGFR).

The method may be used to treat an individual having or suspected of having cancer, such as, for example, solid cancers (e.g., tumors). For example, the cancer may be lung cancer, such as, for example, Mutant EGFR non-small cell lung cancer. The non-small cell lung cancer may have one or more of the following mutations: L858R, exon19del (e.g., delE756-A750, delL747-A750insP, delL747-T751), T790M, and/or C797S. In other embodiments, the cancer may also be breast cancer, such as, for example, Her2 over-expressing breast cancer. Treatment may be reduction in size of a tumor, partial tumor eradication, or completely tumor eradication. A method may be used in combination with other known cancer therapies.

In various embodiments, the administration of the compound or the composition comprising the compound may be oral, parenteral, sublingual, transdermal, rectal, transmucosal, topical, inhaled, or buccal administration, or combinations thereof; and wherein the parenteral administration comprises intravenous, intraarterial, intracranial, intradermal, subcutaneous, intraperitoneal, intramuscular, intrathecal, or intraarticular administration.

In another aspect of the present disclosure, a kit is provided, comprising a composition comprising the compound of the disclosure, or constituents to prepare a composition comprising the compound of the disclosure.

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The following Statements provide various embodiments of the disclosure, they are not intended to be limiting.
Statement 1. A compound having the following structure:

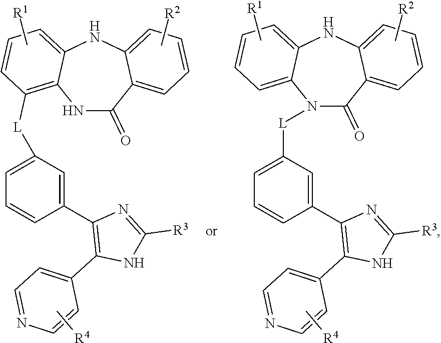

wherein R¹ is a halogen, a substituted aliphatic group, an unsubstituted aliphatic group, or is absent; R² is a halogen, a substituted aliphatic group, an unsubstituted aliphatic group, or is absent; R³ is a substituted aliphatic group; an unsubstituted aliphatic group; a thioether group, or absent; R⁴ is an amide or a secondary amine; and L is a linker.
Statement 2. The compound according to Statement 1, wherein
   R¹ is —I, —F, —Br, —Cl, a substituted or unsubstituted alkyl group, or absent;
   R² is —I, —F, —Br, —Cl, a substituted or unsubstituted alkyl group, or absent;
   R³ is

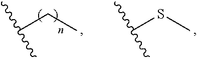

or absent, wherein n is 0, 1, 2, or 3;
R⁴ is

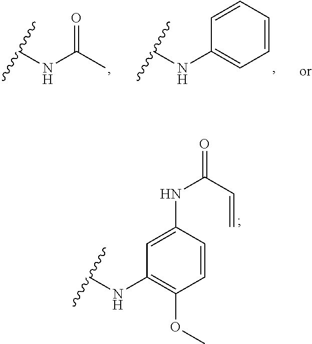

and/or
L is

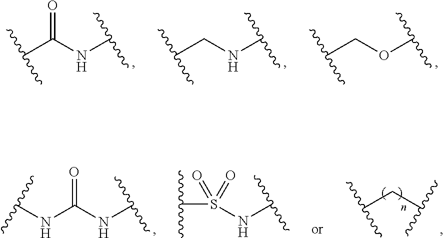

wherein n is 1, 2, or 3.
Statement 3. The compound according to Statement 1 or Statement 2, wherein the compound has the following structure:

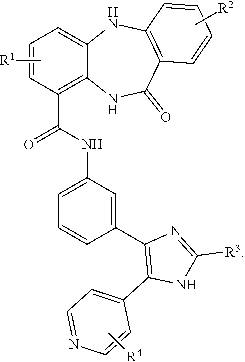

Statement 4. The compound according to any one of the preceding Statements, wherein the compound has the following structure:

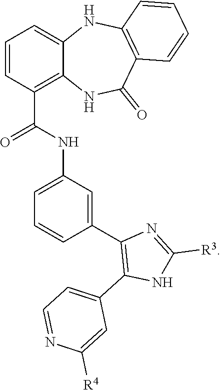

Statement 5. The compound according to Statement 1 or Statement 2, wherein the compound has the following structure:

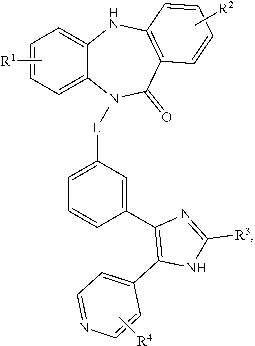

wherein L is

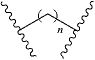

and n is 1, 2, or 3.
Statement 6. The compound according to Statement 1 or Statement 2, wherein the compound has the following structure:

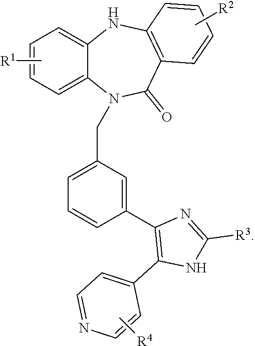

Statement 7. The compound according to Statement 1 or Statement 2, wherein the compound has the following structure:

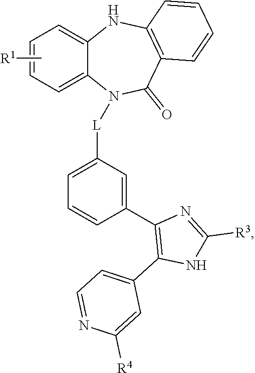

wherein L is

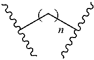

and n is 1, 2, or 3.
Statement 8. The compound according to Statement 1 or Statement 2, wherein the compound has the following structure:

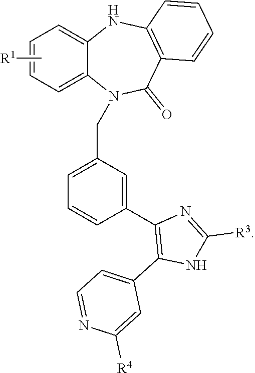

Statement 9. The compound according to according to Statement 1 or Statement 2, wherein the compound has the following structure:

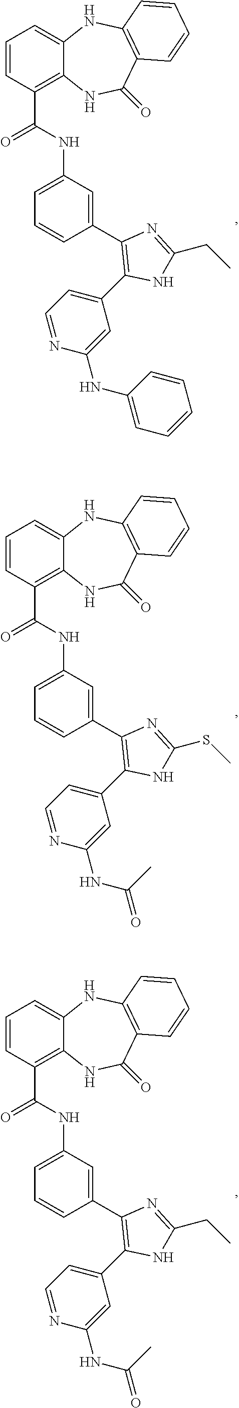 [SMILES: CCc1nc(-c2cccc(NC(=O)c3cccc4c3NC(=O)c3ccccc3N4)c2)c(-c2ccnc(Nc3ccccc3)c2)[nH]1 ; CSc1nc(-c2cccc(NC(=O)c3cccc4c3NC(=O)c3ccccc3N4)c2)c(-c2ccnc(NC(C)=O)c2)[nH]1 ; CCc1nc(-c2cccc(NC(=O)c3cccc4c3NC(=O)c3ccccc3N4)c2)c(-c2ccnc(NC(C)=O)c2)[nH]1]

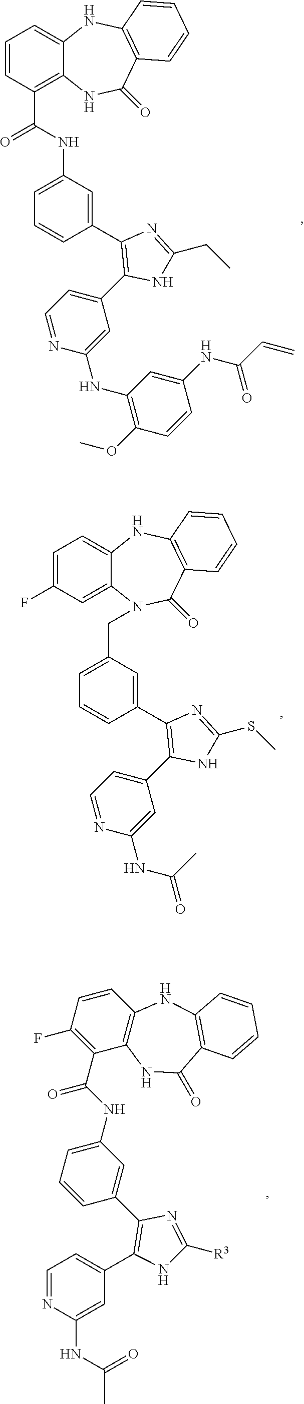

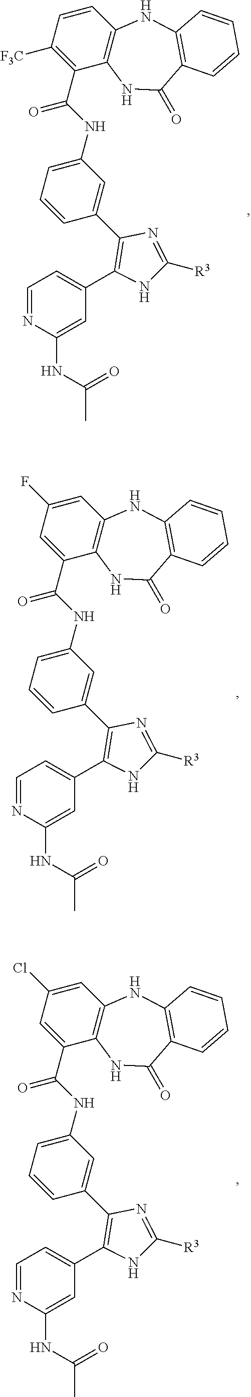

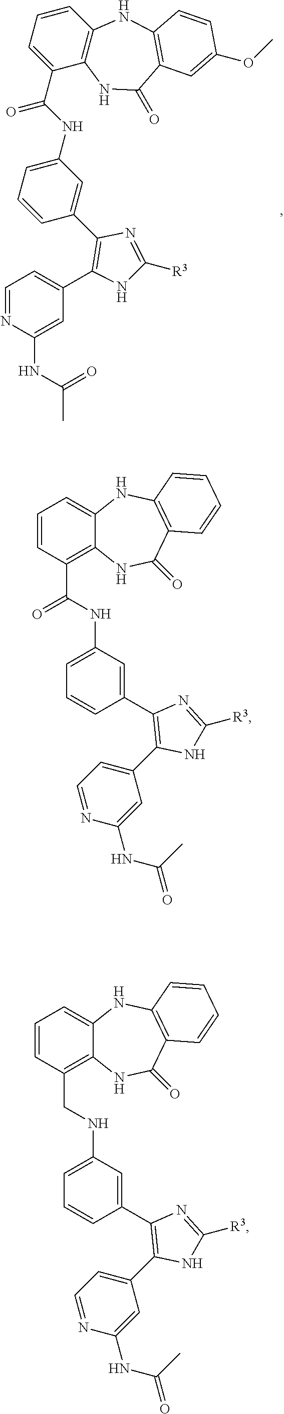

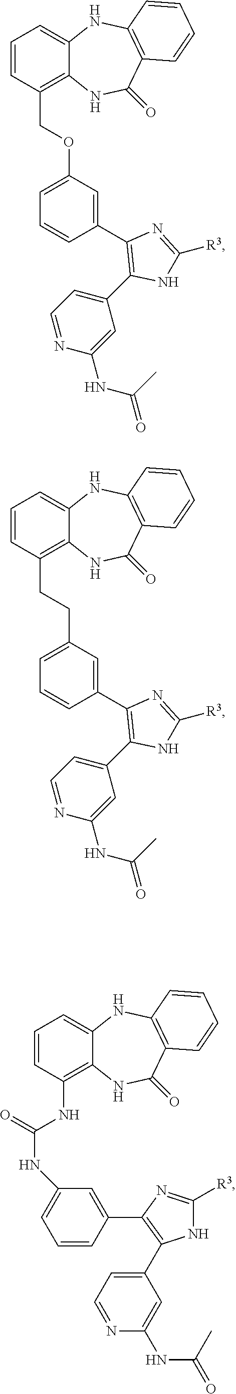

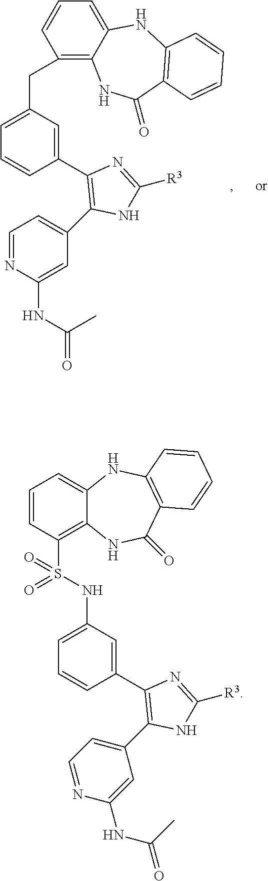

Statement 10. A composition comprising the compound according to any one of the preceding Statements.
Statement 11. The composition according to Statement 10, further comprising a pharmaceutically acceptable carrier.
Statement 12. A method of treating an individual having or suspected of having cancer, comprising: administering a therapeutically effective amount of the compound according to any one of Statements 1 to 9 or a composition comprising one or more compounds according to any one of Statements 1 to 9 to the individual.
Statement 13. The method according to Statement 12, wherein the individual is a human or non-human animal.
Statement 14. The method according to Statement 12 or Statement 13, wherein the composition inhibits a mutant epidermal growth factor receptor (EGFR).
Statement 15. The method according to any one of Statements 12 to 14, wherein the cancer is lung cancer or breast cancer.
Statement 16. The method according to any one of Statements 12 to 15, wherein the lung cancer is an EGFR non-small cell lung cancer.
Statement 17. The method according to Statement 16, wherein the non-small cell lung cancer has one or more of the following mutations: L858R, exon19del (e.g., delE756-A750, delL747-A750insP, or delL747-T751), T790M, and/or C797S.
Statement 18. The method according to Statement 15, wherein the breast cancer is HER2 over-expressing breast cancer.
Statement 19. A kit, comprising: a composition comprising the compound according to any one of Statements 1 to 9 or constituents to prepare a composition comprising the compound according to any one of Statements 1 to 9.

The following examples are presented to illustrate the present disclosure. They are not intended to be limiting in any matter.

### Example 1

The following example provides examples of compounds of the present disclosure and uses thereof.

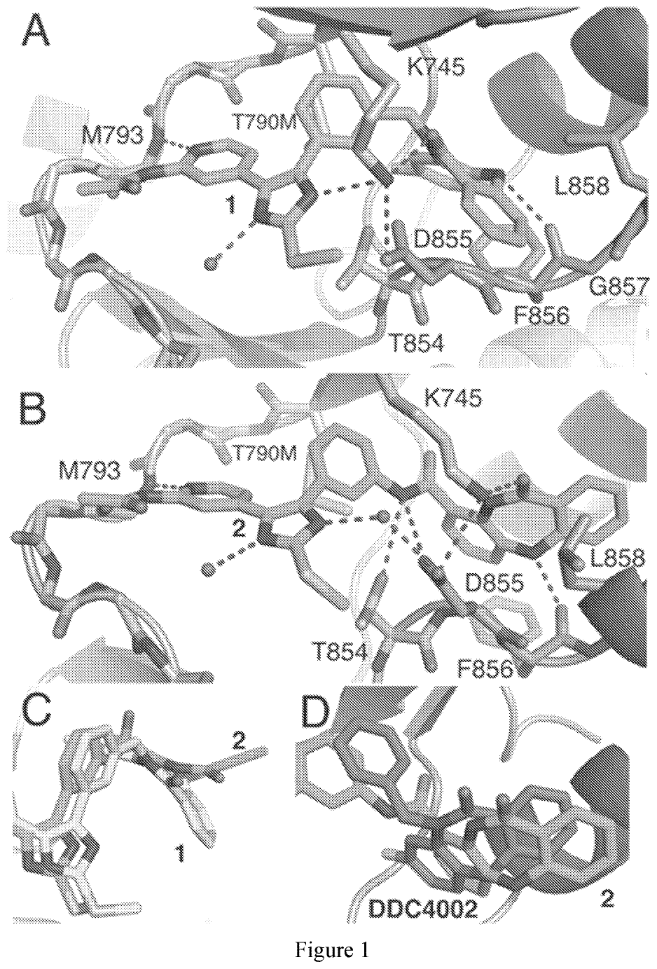

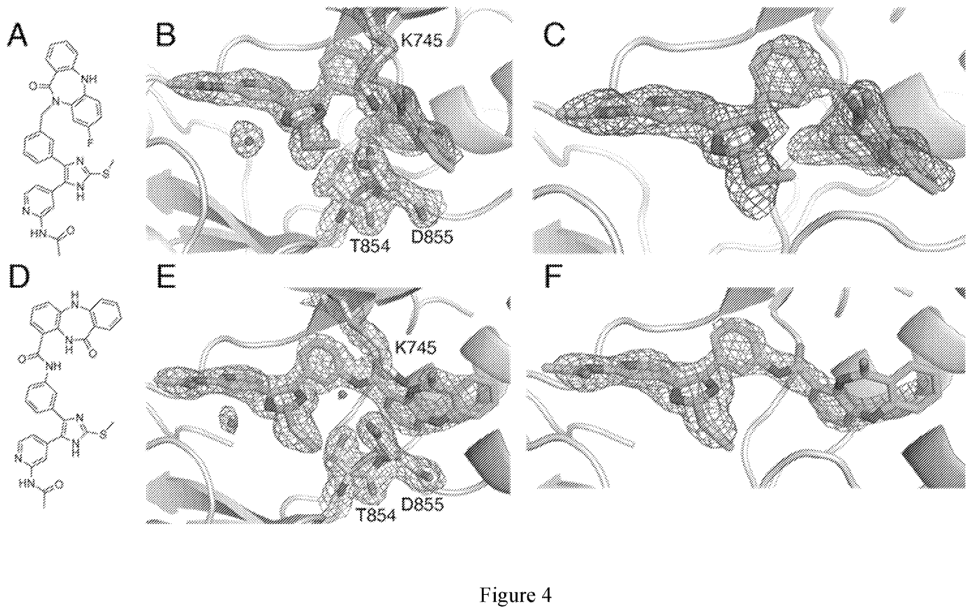

To characterize the binding modes of the N-linked 1 and C-linked 2, soaked X-ray cocrystal structures were used with EGFR(T790M/V948R) which reliably crystallizes in the inactive kinase conformation. A 2.1 Å-resolution cocrystal structure shows 1 bound within the ATP and allosteric sites (FIG. 1A, FIG. 4, Table 1). The imidazole moiety is bound at the ATP site as consistent with earlier structures, while the benzo within the allosteric pocket of 1 is bound in an “outward” pucker (FIG. 1B-C, FIG. 4B-C). Distinctly, a 2.2 Å-resolution cocrystal structure of 2 indicates that the benzo group within the allosteric pocket site binds in the opposite “inward” pucker (Table 2). Another difference pertains to the amide group in 2, which is observed making several H-bonds with T854 and D855 otherwise not possible with 1. A “swing” of K745 toward the benzo ketone was observed in 2, opening a position on the imidazole, which now binds a solvent water (FIG. 1B). These two cocrystal structures show that the alternative linkers in 1 and 2 can influence the conformation of the benzo group at the allosteric pocket (FIG. 1C) and allow for distinctive interactions within the EGFR kinase domain.

TABLE 1
Data collection and refinement statistics. Statistics for
the highest-resolution shell are shown in parentheses.

| | 1 (LN4503) | | 2 (LN5993) | |
|---|---|---|---|---|
| | PDB ID 8FV3 | | PDB ID 8FV4 | |
| Wavelength | 0.97933 | | 0.97918 | |
| Resolution range | 65.8-2.1 | (2.175-2.1) | 60.3-2.2 | (2.279-2.2) |
| Space group | P 1 21 1 | | P 1 21 1 | |
| Unit cell | 72.3699 103.224 | | 70.6064 100.485 | |
| | 87.1399 90 101.48 90 | | 87.3183 90 102.323 90 | |
| Total reflections | 495294 | (49704) | 202025 | (20491) |
| Unique reflections | 73258 | (7323) | 59558 | (5992) |
| Multiplicity | 6.8 | (6.8) | 3.4 | (3.4) |
| Completeness (%) | 99.92 | (99.81) | 98.33 | (98.73) |
| Mean I/sigma(I) | 7.05 | (0.82) | 9.73 | (1.04) |
| Wilson B-factor | 30.77 | | 41.55 | |
| R-merge | 0.1376 | (0.7708) | 0.0732 | (0.4283) |
| R-meas | 0.1491 | (0.8344) | 0.08684 | (0.5079) |
| R-pim | 0.05685 | (0.3169) | 0.04616 | (0.2697) |
| CC½ | 0.996 | (0.868) | 0.997 | (0.91) |
| CC* | 0.999 | (0.964) | 0.999 | (0.976) |
| Reflections used | 73205 | (7310) | 59494 | (5975) |
| in refinement | | | | |
| Reflections used | 1994 | (198) | 1995 | (200) |
| for R-free | | | | |
| R-work | 0.1867 | (0.2433) | 0.2273 | (0.3149) |
| R-free | 0.2285 | (0.3240) | 0.2872 | (0.3644) |
| CC(work) | 0.951 | (0.880) | 0.953 | (0.806) |
| CC(free) | 0.915 | (0.642) | 0.906 | (0.732) |

| | | |
|---|---|---|
| Number of non- | 9881 | 9620 |
| hydrogen atoms | | |
| macromolecules | 9418 | 9319 |
| ligands | 137 | 106 |
| solvent | 326 | 195 |
| Protein residues | 1193 | 1186 |
| RMS(bonds) | 0.008 | 0.008 |
| RMS(angles) | 0.96 | 0.96 |
| Ramachandran | 96.24 | 95.45 |
| favored (%) | | |
| Ramachandran | 2.90 | 3.60 |
| allowed (%) | | |
| Ramachandran | 0.85 | 0.94 |
| outliers (%) | | |
| Rotamer outliers (%) | 1.29 | 1.32 |
| Clashscore | 5.95 | 7.77 |
| Average B-factor | 36.62 | 41.72 |
| macromolecules | 36.57 | 41.73 |
| ligands | 38.50 | 42.34 |
| solvent | 37.06 | 40.99 |

The biochemical potencies of these molecules were characterized in HTRF-based activity assays with purified EGFR kinase domains (Table 2). Strikingly, the N-linked 1 is limitedly potent against WT and mutant EGFR with IC₅₀ values>1 μM while the C-linked inhibitors 2-3 show substantial lower IC₅₀ values 1.2-1.5 nM for LR and 51-64 μM for LRTM and LRTMCS. Time-dependent kinetic parameters for the covalent analogue 4 were obtained with Sox-based reagents, and k_{inact}/K_{I} values show that this molecule is most potent against LR (Table 3). These results indicate that the C-linked 2-3 are far better suited for binding and inhibiting EGFR compared to the N-linked 1, which can be mainly attributed to the structure of the linker connecting the ATP and allosteric sites.

TABLE 2
Reversible Inhibitor Biochemical EGFR IC₅₀ values(nM)
against wt and mutant EGFR kinase domains.

| Compound | wt | LR | LRTM | LRTMCS |
|---|---|---|---|---|
| 1 | >10000 | 1300 ± 100 | >10000 | >10000 |
| 2 | <10 | 1.5 ± 0.1 | 0.059 ± 0.005 | 0.064 ± 0.004 |
| 3 | <10 | 1.2 ± 0.09 | 0.051 ± 0.005 | 0.063 ± 0.005 |
| 4 | —^{[c]} | —^{[c]} | —^{[c]} | 1.8 ± 0.3 |
| 5 | <10 | 1.1 ± 0.2 | 0.87 ± 0.09 | 1.0 ± 0.1 |
| DDC4002^{[a]} | >1000 | 690 ± 120 | 39 ± 4 | 59 ± 8 |
| EAI002^{[a]} | >1000 | n.d. | 52 | n.d. |
| LN2057^{[b]} | —^{[c]} | —^{[c]} | —^{[c]} | 130 ± 40 |

^{[a]}from De Clercq and Heppner et al., ACS Med Chem Letts 2019
^{[b]}from Wittlinger and Heppner et al., J. Med. Chem. 2022
^{[c]}Left blank since these compounds are time-dependent covalent inhibitors of these enzymes.
WT <10 nM due to enzyme concentration being 10 nM.

TABLE 3
Time dependent inhibition of EGFR by 4

| 4 | wt | LR | LRTM |
|---|---|---|---|
| k_{inact}/K_{I} (M − 1s − 1) | 2500 ± 10 | 14100 ± 300 | 1070 ± 40 |
| k_{inact} (min − 1) | 0.18 ± 0.004 | 0.52 ± 0.02 | 0.10 ± 0.004 |
| K_{I} (μM) | 1.20 ± 0.04 | 0.61 ± 0.03 | 1.6 ± 0.1 |

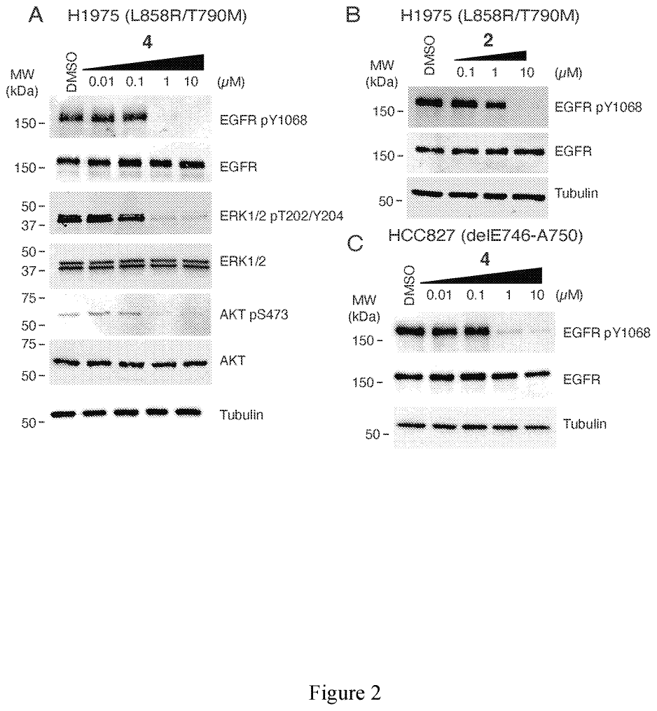

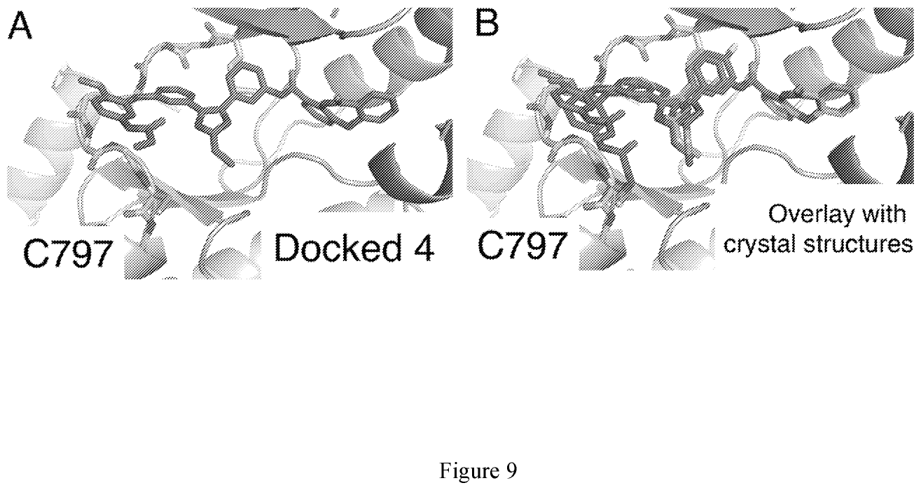

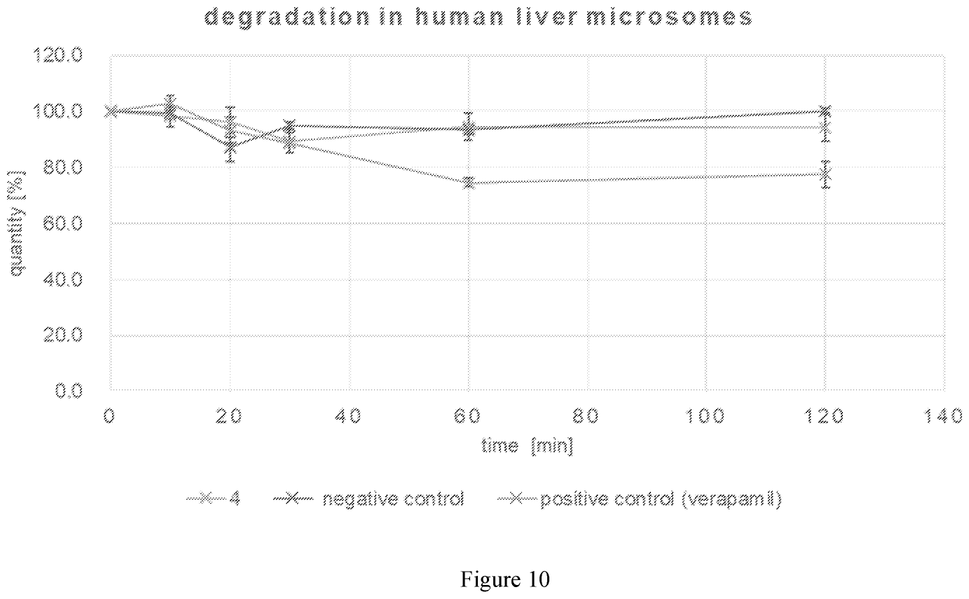

Next evaluated was the biological activity of the C-linked bivalent compounds in human cancer cells. The covalent analogue 4 is most effective in suppression of active EGFR (pY1068) as well as downstream pERK in the NSCLC cell line H1975 (FIG. 2A). Antiproliferative activity experiments in Ba/F3 cells are in line with this observation, showing ~200 nM and ~750 nM potency of 4 against L858R and L858R/T790M, respectively (Table 4). The reversible binding 2 is less effective in H1975 (FIG. 2B) and Ba/F3 cells likely due to the importance of forming a covalent bond in driving cellular activity. It was confirmed that 4 is similarly active in NSCLC HCC827 cells driven by the del(E746-A750) exon19del mutation (FIG. 2C). This observation is informative as EGFR allosteric inhibitors are often limitedly effective against exon19del mutants indicating the bivalent 4 is more closely related to ATP-competitive inhibitors despite extensive binding within the allosteric pocket (FIG. 9). It was observed that 4 is selective against EGFR in the kinome (Table 5) with S (35)=0.084. Further, it was confirmed that this molecule is metabolically stable in liver microsome assays (FIG. 10). The biological activity and early-phase medicinal chemistry properties of 4 demonstrates that our linker optimization of bivalent EGFR inhibitors is a viable route to producing lead compounds for future optimization and pre-clinical evaluation against important activating EGFR mutations.

TABLE 4
Anti-proliferative activities on the proliferation of Ba/F3
cell lines of wild-type EGFR, selected mutants and HER2.
EGFR EC₅₀ ± SE (nM)

| Compound | wt | LR | LR/TM | HER2 |
|---|---|---|---|---|
| 2 | 5100 ± 2000 | 5300 ± 900 | >10000 | n.d. |
| 4 | 950 ± 200 | 220 ± 80 | 770 ± 390 | 1930 ± 1010 |
| DDC4002^{[a]} | >10,000 | >10,000 | >10,000 | — |
| | (9700 ± 500)^{[b]} | (>10,000)^{[b]} | (1500 ± 400)^{[b]} | |
| LN2057 | 20 ± 6 ^{[c]} | <1 ^{[c, d]} | 22 ± 1 ^{[c]} | 2.7 ± 1 ^{[e]} |
| Osimertinib | 110 ± 40 ^{[c]} | 3.3 ± 0.6 ^{[c]} | 8 ± 0 ^{[c]} | 34 ± 8 ^{[e]} |

^{[a, b]}from De Clercq and Heppner et al., ACS Med Chem Letts 2019
^{[b]}values indicate experiments that include cetuximab from
^{[c]} Wittlinger and Heppner et al., J. Med. Chem. 2022
^{[d]} Values below the resolution limit of the assay.)
^{[e]} Heppner et al., ACS Med Chem Letts 2022.

The disclosed bivalent EGFR inhibitors that exhibit a wide range of biochemical IC₅₀ values based on differences in the linker that bridges the allosteric and ATP sites. These bivalent inhibitors are unique among kinase inhibitors due to gatekeeper proximal linkers driving variable potencies and binding conformation within allosteric sites. Aided by X-ray cocrystal structures of compounds exhibiting low versus high potencies, the described compounds demonstrate fragment linking or merging design criteria. Future studies of diverse linker structures and binding modes in kinase or other receptor model systems will enable needed molecular-level understandings to access new starting points more swiftly in FBDD and drug development, and potentially open new avenues in classically undruggable targets.

TABLE 5
Selectivity screening data was obtained from KINOMEscan ™ using
1 μM of compound 4 against 468 kinases. Eurofins DiscoverX
Corporation (San Diego), CA 92121, USA.

| Target | POC |
|---|---|
| AAK1 | 100 |
| ABL1(E255K)-phosphorylated | 65 |
| ABL1(F317I)-nonphosphorylated | 40 |
| ABL1(F317I)-phosphorylated | 84 |
| ABL1(F317L)-nonphosphorylated | 37 |
| ABL1(F317L)-phosphorylated | 85 |
| ABL1(H396P)-nonphosphorylated | 97 |
| ABL1(H396P)-phosphorylated | 74 |
| ABL1(M351T)-phosphorylated | 67 |
| ABL1(Q252H)-nonphosphorylated | 100 |
| ABL1(Q252H)-phosphorylated | 90 |
| ABL1(T315I)-nonphosphorylated | 98 |
| ABL1(T315I)-phosphorylated | 100 |
| ABL1(Y253F)-phosphorylated | 72 |
| ABL1-nonphosphorylated | 100 |
| ABL1-phosphorylated | 80 |
| ABL2 | 92 |
| ACVR1 | 96 |
| ACVR1B | 85 |
| ACVR2A | 77 |
| ACVR2B | 72 |
| ACVRL1 | 75 |
| ADCK3 | 100 |
| ADCK4 | 85 |
| AKT1 | 89 |
| AKT2 | 91 |
| AKT3 | 100 |
| ALK | 100 |
| ALK(C1156Y) | 87 |
| ALK(L1196M) | 89 |
| AMPK-alpha1 | 81 |
| AMPK-alpha2 | 56 |
| ANKK1 | 87 |
| ARK5 | 100 |
| ASK1 | 100 |
| ASK2 | 100 |
| AURKA | 90 |
| AURKB | 100 |
| AURKC | 100 |
| AXL | 98 |
| BIKE | 100 |
| BLK | 1.7 |
| BMPR1A | 85 |
| BMPR1B | 100 |
| BMPR2 | 88 |
| BMX | 66 |
| BRAF | 90 |
| BRAF(V600E) | 100 |
| BRK | 96 |
| BRSK1 | 100 |
| BRSK2 | 93 |
| BTK | 54 |
| BUB1 | 100 |
| CAMK1 | 81 |
| CAMK1B | 94 |
| CAMK1D | 100 |
| CAMK1G | 100 |
| CAMK2A | 65 |
| CAMK2B | 72 |
| CAMK2D | 88 |
| CAMK2G | 100 |
| CAMK4 | 99 |
| CAMKK1 | 85 |
| CAMKK2 | 95 |
| CASK | 97 |
| CDC2L1 | 100 |
| CDC2L2 | 91 |
| CDC2L5 | 74 |
| CDK11 | 100 |
| CDK2 | 95 |
| CDK3 | 100 |
| CDK4 | 100 |
| CDK4-cyclinD1 | 94 |
| CDK4-cyclinD3 | 49 |
| CDK5 | 100 |
| CDK7 | 49 |
| CDK8 | 81 |
| CDK9 | 100 |
| CDKL1 | 100 |
| CDKL2 | 100 |
| CDKL3 | 100 |
| CDKL5 | 98 |
| CHEK1 | 100 |
| CHEK2 | 87 |
| CIT | 6.3 |
| CLK1 | 100 |
| CLK2 | 63 |
| CLK3 | 74 |
| CLK4 | 100 |
| CSF1R | 100 |
| CSF1R-autoinhibited | 69 |
| CSK | 14 |
| CSNK1A1 | 56 |
| CSNK1A1L | 91 |
| CSNK1D | 87 |
| CSNK1E | 88 |
| CSNK1G1 | 93 |
| CSNK1G2 | 91 |
| CSNK1G3 | 98 |
| CSNK2A1 | 100 |
| CSNK2A2 | 58 |
| CTK | 54 |
| DAPK1 | 86 |
| DAPK2 | 100 |
| DAPK3 | 62 |
| DCAMKL1 | 100 |
| DCAMKL2 | 100 |
| DCAMKL3 | 86 |
| DDR1 | 90 |
| DDR2 | 100 |
| DLK | 100 |
| DMPK | 45 |
| DMPK2 | 0 |
| DRAK1 | 84 |
| DRAK2 | 94 |
| DYRK1A | 100 |
| DYRK1B | 88 |
| DYRK2 | 75 |
| EGFR | 17 |
| EGFR(E746-A750del) | 13 |
| EGFR(G719C) | 6.5 |
| EGFR(G719S) | 13 |
| “EGFR(L747-E749del + A750P)” | 8 |
| “EGFR(L747-S752del + P753S)” | 16 |
| “EGFR(L747-T751del + Sins)” | 7.6 |
| EGFR(L858R) | 5 |
| “EGFR(L858R + T790M)” | 8.3 |
| EGFR(L861Q) | 2.7 |
| EGFR(S752-1759del) | 6 |
| EGFR(T790M) | 12 |
| EIF2AK1 | 75 |
| EPHA1 | 100 |
| EPHA2 | 100 |
| EPHA3 | 81 |
| EPHA4 | 95 |
| EPHA5 | 100 |
| EPHA6 | 90 |
| EPHA7 | 100 |
| EPHA8 | 98 |
| EPHB1 | 92 |
| EPHB2 | 100 |
| EPHB3 | 100 |
| EPHB4 | 100 |
| EPHB6 | 100 |
| ERBB2 | 18 |
| ERBB3 | 84 |
| ERBB4 | 4.9 |
| ERK1 | 99 |
| ERK2 | 100 |
| ERK3 | 100 |
| ERK4 | 100 |
| ERK5 | 100 |
| ERK8 | 100 |
| ERN1 | 74 |
| FAK | 99 |
| FER | 36 |
| FES | 28 |
| FGFR1 | 86 |
| FGFR2 | 100 |
| FGFR3 | 94 |
| FGFR3(G697C) | 100 |
| FGFR4 | 100 |
| FGR | 19 |
| FLT1 | 100 |
| FLT3 | 100 |
| FLT3(D835H) | 92 |
| FLT3(D835V) | 100 |
| FLT3(D835Y) | 96 |
| FLT3(ITD) | 100 |
| “FLT3(ITD + D835V)” | 100 |
| “FLT3(ITD + F691L)” | 79 |
| FLT3(K663Q) | 87 |
| FLT3(N841I) | 97 |
| FLT3(R834Q) | 100 |
| FLT3-autoinhibited | 100 |
| FLT4 | 100 |
| FRK | 20 |
| FYN | 64 |
| GAK | 16 |
| “GCN2(Kin.Dom.2 + S808G)” | 100 |
| GRK1 | 94 |
| GRK2 | 100 |
| GRK3 | 100 |
| GRK4 | 100 |
| GRK7 | 99 |
| GSK3A | 65 |
| GSK3B | 100 |
| HASPIN | 100 |
| HCK | 14 |
| HIPK1 | 51 |
| HIPK2 | 100 |
| HIPK3 | 100 |
| HIPK4 | 92 |
| HPK1 | 45 |
| HUNK | 32 |
| ICK | 100 |
| IGF1R | 90 |
| IKK-alpha | 100 |
| IKK-beta | 88 |
| IKK-epsilon | 100 |
| INSR | 97 |
| INSRR | 87 |
| IRAK1 | 85 |
| IRAK3 | 39 |
| IRAK4 | 43 |
| ITK | 87 |
| JAK1(JH1domain-catalytic) | 91 |
| JAK1(JH2domain-pseudokinase) | 83 |
| JAK2(JH1domain-catalytic) | 86 |
| JAK3(JH1domain-catalytic) | 100 |
| JNK1 | 85 |
| JNK2 | 78 |
| JNK3 | 53 |
| KIT | 100 |
| KIT(A829P) | 56 |
| KIT(D816H) | 39 |
| KIT(D816V) | 94 |
| KIT(L576P) | 84 |
| KIT(V559D) | 100 |
| KIT(V559D + T670I) | 95 |
| KIT(V559D + V654A) | 93 |
| KIT-autoinhibited | 100 |
| LATS1 | 100 |
| LATS2 | 62 |
| LCK | 7.7 |
| LIMK1 | 79 |
| LIMK2 | 93 |
| LKB1 | 100 |
| LOK | 49 |
| LRRK2 | 93 |
| LRRK2(G2019S) | 100 |
| LTK | 48 |
| LYN | 22 |
| LZK | 90 |
| MAK | 89 |
| MAP3K1 | 100 |
| MAP3K15 | 74 |
| MAP3K2 | 100 |
| MAP3K3 | 51 |
| MAP3K4 | 100 |
| MAP4K2 | 36 |
| MAP4K3 | 9.5 |
| MAP4K4 | 6.1 |
| MAP4K5 | 2.5 |
| MAPKAPK2 | 100 |
| MAPKAPK5 | 100 |
| MARK1 | 92 |
| MARK2 | 85 |
| MARK3 | 100 |
| MARK4 | 92 |
| MAST1 | 100 |
| MEK1 | 100 |
| MEK2 | 100 |
| MEK3 | 100 |
| MEK4 | 100 |
| MEK5 | 41 |
| MEK6 | 73 |
| MELK | 100 |
| MERTK | 83 |
| MET | 69 |
| MET(M1250T) | 89 |
| MET(Y1235D) | 97 |
| MINK | 10 |
| MKK7 | 31 |
| MKNK1 | 100 |
| MKNK2 | 100 |
| MLCK | 86 |
| MLK1 | 86 |
| MLK2 | 97 |
| MLK3 | 97 |
| MRCKA | 0 |
| MRCKB | 1.7 |
| MST1 | 6 |
| MST1R | 85 |
| MST2 | 14 |
| MST3 | 73 |
| MST4 | 94 |
| MTOR | 100 |
| MUSK | 100 |
| MYLK | 84 |
| MYLK2 | 84 |
| MYLK4 | 95 |
| MYO3A | 14 |
| MYO3B | 31 |
| NDR1 | 73 |
| NDR2 | 77 |
| NEK1 | 90 |
| NEK10 | 86 |
| NEK11 | 100 |
| NEK2 | 98 |
| NEK3 | 100 |
| NEK4 | 100 |
| NEK5 | 100 |
| NEK6 | 100 |
| NEK7 | 86 |
| NEK9 | 83 |
| NIK | 100 |
| NIM1 | 78 |
| NLK | 48 |
| OSR1 | 89 |
| p38-alpha | 46 |
| p38-beta | 59 |
| p38-delta | 100 |
| p38-gamma | 100 |
| PAK1 | 100 |
| PAK2 | 100 |
| PAK3 | 70 |
| PAK4 | 89 |
| PAK6 | 93 |
| PAK7 | 77 |
| PCTK1 | 100 |
| PCTK2 | 100 |
| PCTK3 | 93 |
| PDGFRA | 69 |
| PDGFRB | 100 |
| PDPK1 | 84 |
| PFCDPK1(P. falciparum) | 87 |
| PFPK5(P. falciparum) | 100 |
| PFTAIRE2 | 84 |
| PFTK1 | 90 |
| PHKG1 | 96 |
| PHKG2 | 73 |
| PIK3C2B | 74 |
| PIK3C2G | 50 |
| PIK3CA | 70 |
| PIK3CA(C420R) | 66 |
| PIK3CA(E542K) | 100 |
| PIK3CA(E545A) | 100 |
| PIK3CA(E545K) | 100 |
| PIK3CA(H1047L) | 100 |
| PIK3CA(H1047Y) | 66 |
| PIK3CA(I800L) | 100 |
| PIK3CA(M1043I) | 55 |
| PIK3CA(Q546K) | 100 |
| PIK3CB | 90 |
| PIK3CD | 71 |
| PIK3CG | 65 |
| PIK4CB | 93 |
| PIKFYVE | 94 |
| PIM1 | 94 |
| PIM2 | 88 |
| PIM3 | 100 |
| PIP5K1A | 75 |
| PIP5K1C | 78 |
| PIP5K2B | 100 |
| PIP5K2C | 66 |
| PKAC-alpha | 100 |
| PKAC-beta | 98 |
| PKMYT1 | 99 |
| PKN1 | 88 |
| PKN2 | 100 |
| PKNB(M. tuberculosis) | 97 |
| PLK1 | 50 |
| PLK2 | 100 |
| PLK3 | 87 |
| PLK4 | 71 |
| PRKCD | 55 |
| PRKCE | 100 |
| PRKCH | 69 |
| PRKCI | 100 |
| PRKCQ | 18 |
| PRKD1 | 42 |
| PRKD2 | 62 |
| PRKD3 | 67 |
| PRKG1 | 100 |
| PRKG2 | 62 |
| PRKR | 87 |
| PRKX | 100 |
| PRP4 | 46 |
| PYK2 | 100 |
| QSK | 100 |
| RAF1 | 73 |
| RET | 99 |
| RET(M918T) | 99 |
| RET(V804L) | 99 |
| RET(V804M) | 91 |
| RIOK1 | 100 |
| RIOK2 | 83 |
| RIOK3 | 100 |
| RIPK1 | 100 |
| RIPK2 | 62 |
| RIPK4 | 100 |
| RIPK5 | 79 |
| ROCK1 | 100 |
| ROCK2 | 94 |
| ROS1 | 80 |
| RPS6KA4(Kin.Dom.1-N-terminal) | 96 |
| RPS6KA4(Kin.Dom.2-C-terminal) | 75 |
| RPS6KA5(Kin.Dom.1-N-terminal) | 83 |
| RPS6KA5(Kin.Dom.2-C-terminal) | 100 |
| RSK1(Kin.Dom.1-N-terminal) | 4.7 |
| RSK1(Kin.Dom.2-C-terminal) | 75 |
| RSK2(Kin.Dom.1-N-terminal) | 100 |
| RSK2(Kin.Dom.2-C-terminal) | 100 |
| RSK3(Kin.Dom.1-N-terminal) | 97 |
| RSK3(Kin.Dom.2-C-terminal) | 80 |
| RSK4(Kin.Dom.1-N-terminal) | 100 |
| RSK4(Kin.Dom.2-C-terminal) | 80 |
| S6K1 | 100 |
| SBK1 | 84 |
| SGK | 86 |
| SgK110 | 100 |
| SGK2 | 83 |
| SGK3 | 90 |
| SIK | 100 |
| SIK2 | 100 |
| SLK | 100 |
| SNARK | 100 |
| SNRK | 82 |
| SRC | 73 |
| SRMS | 61 |
| SRPK1 | 100 |
| SRPK2 | 100 |
| SRPK3 | 100 |
| STK16 | 100 |
| STK33 | 100 |
| STK35 | 69 |
| STK36 | 30 |
| STK39 | 85 |
| SYK | 63 |
| TAK1 | 95 |
| TAOK1 | 100 |
| TAOK2 | 100 |
| TAOK3 | 95 |
| TBK1 | 95 |
| TEC | 45 |
| TESK1 | 94 |
| TGFBR1 | 75 |
| TGFBR2 | 75 |
| TIE1 | 100 |
| TIE2 | 71 |
| TLK1 | 87 |
| TLK2 | 87 |
| TNIK | 33 |
| TNK1 | 22 |
| TNK2 | 3.8 |
| TNNI3K | 89 |
| TRKA | 77 |
| TRKB | 100 |
| TRKC | 87 |
| TRPM6 | 99 |
| TSSK1B | 95 |
| TSSK3 | 100 |
| TTK | 72 |
| TXK | 42 |
| TYK2(JH1domain-catalytic) | 91 |
| TYK2(JH2domain-pseudokinase) | 100 |
| TYRO3 | 32 |
| ULK1 | 100 |
| ULK2 | 80 |
| ULK3 | 100 |
| VEGFR2 | 100 |
| VPS34 | 84 |
| VRK2 | 100 |
| WEE1 | 85 |
| WEE2 | 98 |
| WNK1 | 100 |
| WNK2 | 62 |
| WNK3 | 78 |
| WNK4 | 85 |
| YANK1 | 84 |
| YANK2 | 63 |
| YANK3 | 96 |
| YES | 20 |
| YSK1 | 76 |
| YSK4 | 88 |
| ZAK | 87 |
| ZAP70 | 97 |

### Example 2

Described herein are synthesis and characterization of various embodiments of the present disclosure.

The following are experimental procedures.

Protein expression and purification. The EGFR kinase domain (residues 696-1022) was cloned into pTriEx with an N-terminal 6×His-glutathione S-transferase (GST) fusion tag followed by a TEV protease cleavage site. EGFR WT, L858R, L858R/T790M, L858R/T790M/C797S was expressed after baculoviral infection in SF9 cells and EGFR(T790M/V948R) was expressed in SF21 cells. Briefly, cells were pelleted and resuspended in lysis buffer composed of 50 mM Tris pH 8.0, 500 mM NaCl, 1 mM tris(2-carboxyethyl)phosphine (TCEP), and 5% glycerol. Cells were lysed via sonication prior to ultracentrifugation at >200,000 g for 1 h. Imidazole pH 8.0 was added to the supernatant for a final concentration of 40 mM and flowed through a column containing Ni-NTA agarose beads. The resin was washed with lysis buffer supplemented with 40 mM imidazole and eluted with lysis buffer containing 200 mM imidazole. Eluted EGFR kinase domain was dialyzed overnight in the presence of 5% (w/w) TEV protease against dialysis buffer containing 50 mM Tris pH 8.0, 500 mM NaCl, 1 mM TCEP, and 5% glycerol. The cleaved protein was passed through Ni-NTA resin to remove the 6×His-GST fusion protein and TEV prior to size exclusion chromatography on a prep-grade Superdex S200 column in 50 mM Tris pH 8.0, 500 mM NaCl, 1 mM TCEP, and 5% glycerol. Fractions containing EGFR kinase of >95% purity as assessed by Coomassie-stained SDS-PAGE were concentrated to approximately 4 mg/mL as determined by Bradford assay or absorbance.

Crystallization and structure determination. EGFR(T790M/V948R) pre-incubated with 1 mM AMP-PNP and 10 mM MgCl₂ on ice was prepared by hanging-drop vapor diffusion over a reservoir solution containing 0.1 M Bis-Tris (pH=5.5), 25% PEG-3350, and 5 mM TCEP (buffer A for crystals soaked with compound 1) or 0.1 M Bis-Tris (pH=5.7), 30% PEG-3350 TCEP (buffer B for crystals soaked with compound 2). Drops containing crystals in buffer A and B were exchanged with solutions of both buffers containing ~1.0 mM 1-51 or 2 three times for an hour and then left overnight. Crystals were flash frozen after rapid immersion in a cryoprotectant solution with buffer A or BA containing 25% ethylene glycol. X-ray diffraction data of T790M/V948R-compound 2 crystals was collected at 100K at the Advanced Light Source a part of the Northeastern Collaborative Access Team (NE-CAT) on Beamline 24-ID-C. While data on T790M/V948R-compound 1 crystals were collected at 100K at the National Synchrotron Light Source II 17-ID-2.^{[4]} Diffraction data was processed and merged in Xia2 using aimless and dials. The structure was determined by molecular replacement with the program PHASER using the inactive kinase domain EGFR(T790M/V948R) kinase from our previous work excluding the LN3844 ligand (PDB 6WXN). Repeated rounds of manual refitting and crystallographic refinement were performed using COOT and Phenix. The inhibitor was modeled into the closely fitting positive Fₒ-Fₑ electron density and then included in following refinement cycles. Statistics for diffraction data processing and structure refinement are shown in Table S1. Due to a mixture of difference map density with contributions from both AMP-PNP and 2 in in Chain C we elected to leave this chain without bound ligands.

Time-dependent Kinase Inhibition Assays. Biochemical assays were performed with commercially available EGFR WT, cytoplasmic domain (669-1210), GST-tagged, Carna (Cat #/Lot #: 08-115/21CBS-0127H), EGFR [T790M/L858R] and EGFR [L858R], cytoplasmic domain (669-1210), GST-tagged, Carna (Cat #/Lot #: 08-510/12CBS-0765M). Reactions were performed with kinase domain enzyme concentrations of 4 nM in final solutions of 52 mM HEPES pH 7.5, 1 mM ATP, 0.5 mM TCEP, 0.011% Brij-35, 0.25% glycerol, 0.1 mg/ml BSA, 0.52 mM EGTA, 10 mM MgCl₂, 15 μM Sox-based substrate (AQT0734). BSA was not included in this experiment to prevent interference with irreversible inhibitor characterization via off-target binding. All reactions were run for 240 minutes at 30° C. Time-dependent fluorescence from the Sox-based substrate was monitored in PerkinElmer ProxiPlate-384 Plus, white shallow well microplates (Cat. #6008280) Biotek Synergy Neo 2 microplate reader with excitation (360 nm) and emission (485 nm) wavelengths. 2 was dosed between 0 and 10 μM in 24-point curves with 1.5-fold dilutions. Fluorescence, determined with identical reactions but lacking purified enzyme or crude cell lysate was subtracted from the total fluorescence signal for each time point, with both determined in duplicate, to obtain corrected relative fluorescence units (RFU). Corrected RFU values then were plotted vs. time and the reaction velocity for the first ~40 min (initial reaction rates) were determined from the slope using GraphPad Prism (La Jolla, CA) with units of RFU/min.

HTRF Assays. Biochemical assays for EGFR domains were carried out using a homogeneous time-resolved fluorescence (HTRF) KinEASE-TK (Cisbio) assay, as described previously. Assays were optimized for ATP concentration of 100 μM with enzyme concentrations WT EGFR 10 nM, L858R 0.1 nM, L858R/T790M at 0.02 nM and L858R/T790M/C797S at 0.02 nM. Inhibitor compounds in DMSO were dispensed directly in 384-well plates with the D300 digital dispenser (Hewlett Packard) followed immediately by the addition of aqueous buffered solutions using the Multidrop Combi Reagent Dispenser (Thermo Fischer). Compound IC₅₀ values were determined by 11-point inhibition curves (from 10.0 to 0.00130 μM) in triplicate. The data was graphically displayed using GraphPad Prism version 7.0, (GraphPad software). The curves were fitted using a non-linear regression model with a sigmoidal dose response.

Ba/F3 Cellular Antiproliferative Experiments. The parental Ba/F3 cells was a generous gift from the laboratory of Dr. David Weinstock (in 2014), Dr. Pasi Jänne (2020) and was used to generate the wildtype EGFR, wildtype HER2, L858R, and L858R/T790M EGFR mutant Ba/F3 cells. These cells were previously characterized as described. All Ba/F3 cells were cultured in RPMI1640 media with 10% fetal bovine serum and 1% penicillin and streptomycin. All cell lines were tested negative for Mycoplasma using Mycoplasma Plus PCR Primer Set (Agilent) and were passaged and/or used for no longer than 4 weeks for all experiments. Assay reagents were purchased from MilliporeSigma (Cat #R7017-5G). Ba/F3 cells were plated and treated with increasing concentrations of inhibitors in triplicate for 72 hours. Compounds were dispensed using the Tecan D300e Digital Dispenser. Cellular growth or the inhibition of growth was assessed by resazurin viability assay. All experiments were repeated at least 3 times and values were reported as an average of n=3 with standard deviation.

Compound Docking. Computer-aided compound docking was performed with Glide (Schrödinger, LLC, New York, NY, 2021, re. 2021-2) and Maestro 12.8.117. The receptor grid was generated from the EGFR(T790M/V948R) kinase domain from Chain D of PDB ID 8FV4 (compound 2), and ligands were prepared with LigPrep. The best binding poses were ranked based on the lowest docking and glide score values.

Western blotting. H1975 cells were treated for 6 hours with concentrations and inhibitors indicated in the figure legend. Cells were collected and lysed with lysis buffer (5M NaCl, 1M TRIS pH 8.0, 10% SDS, 10% Triton X-100 and a tablet of protease inhibitors) and processed for Western blotting analysis. Primary antibodies used; phospho-EGFR (Tyr1068; #2234, 1:1,000), EGFR (#4267; 1:1,000), phospho-AKT (Ser473; #4060, 1:1,000), AKT (#9272, 1:1,000), phospho-ERK1/2 (Thr202/Tyr204; #4370, 1:1,000), and ERK1/2 (#4695, 1:1,000) antibodies; were purchased from Cell Signaling Technology. Secondary Goat anti-rabbit IgG starbright blue 700 and Anti-tubulin hFAB Rhodamine Tubulin were purchased from Bio-rad.

Metabolic stability in Human Liver Microsomes (HLM). Pooled liver microsomes from humans (male) were purchased from Sekisui XenoTech, LLC, Kansas City, KS, USA. Metabolic stability assays were performed in the presence of an NADPH-regenerating system consisting of 5 mM glucose-6-phosphate, 5 U/mL glucose-6-phosphate dehydrogenase, and 1 mM NADP⁺. Liver microsomes (20 mg/mL), NADPH-regenerating system, and 4 mM MgCl₂·6 H₂O in 0.1 M TRIS-HCl-buffer (pH 7.4) were preincubated for 5 min at 37° C. and 750 rpm on a shaker. The reaction was started by adding the preheated compound at 10 mM resulting in a final concentration of 0.1 mM. The reaction was quenched at selected time points (0, 10, 20, 30, 60, and 120 min) by pipetting 100 μL of internal standard (ketoprofen) at a concentration of 150 μM in acetonitrile. The samples were vortexed for 30 s and centrifuged (21910 relative centrifugal force, 4° C., 20 min). The supernatant was used directly for LC-MS analysis. All compound incubations were conducted at least in triplicates. Additionally, a negative control containing BSA (20 mg/mL) instead of liver microsomes and a positive control using verapamil instead of compound were performed. A limit of 1% organic solvent during incubation was not exceeded. Sample separation and detection were performed on an Alliance 2695 Separations Module HPLC system (Waters Corporation, Milford, MA, USA) equipped with a Phenomenex Kinetex 2.6 μm XB—C18 100 Å 50×3 mm column (Phenomenex Inc., Torrance, CA, USA) coupled to an Alliance 2996 Photodiode Array Detector and a MICROMASS QUATTRO micro API mass spectrometer (both Waters Corporation, Milford, MA, USA) using electrospray ionization in positive mode. Mobile phase A: 90% water, 10% acetonitrile and additionally 0.1% formic acid (v/v), mobile phase B: 100% acetonitrile with additionally 0.1% formic acid (v/v). The gradient was set to: 0-2.5 min 0% B, 2.5-10 min from 0 to 40% B, 10-12 min 40% B, 12.01-15 min from 40 to 0% B at a flow rate of 0.7 mL/min. Samples were maintained at 10° C., the column temperature was set to 20° C. with an injection volume of 5 μL. Spray, cone, extractor, and RF lens voltages were at 4 kV, 30 V, 8 V and 2 V, respectively. The source and desolvation temperatures were set to 120° C. and 350° C., respectively, and the desolvation gas flow was set to 750 L/h. Data analysis was conducted using MassLynx 4.1 software (Waters Corporation, Milford, MA, USA).

General information for chemical synthesis. All starting materials, reagents, and (anhydrous) solvents were commercially available and were used as received without any further purification or drying procedures unless otherwise noted. All NMR spectra were obtained with Bruker Avance 200 MHz and Bruker Avance 400 MHz spectrometers or with a Bruker Avance 600 MHz spectrometer (NMR Department, Institute of Organic Chemistry, Eberhard-Karls-Universität Tübingen). Solvents for NMR are noted in the experimental procedures for each compound. Residual solvent peaks were used to calibrate the chemical shifts. Chemical shifts (δ) are reported in parts per million. Mass spectra were obtained by Advion TLC-MS (ESI) and from the MASS Spectrometry Department (ESI-HRMS), Institute of Organic Chemistry, Eberhard-Karls-Universität Tübingen. The purity of the tested compounds was determined via HPLC analysis on an Agilent 1100 Series LC with a Phenomenex Luna C8 column (150×4.6 mm, 5 μm), and detection was performed with a UV diode array detector (DAD) at 254 and 230 nm wavelengths and was >95%. Elution was carried out with the following gradient: 0.01 M KH₂PO₄, pH 2.30 (solvent A), and MeOH (solvent B), 40% B to 85% B in 8 min, 85% B for 5 min, 85% to 40% B in 1 min, 40% B for 2 min, stop time of 16 min, 5 μL injection volume, flow rate of 1.5 mL/min, and 25° C. oven temperature. Thin-layer chromatography (TLC) analyses were performed on fluorescent silica gel 60 F254 plates (Merck) and visualized via UV illumination at 254 and 366 nm. Column chromatography was performed on Davisil LC60A 20-45 μm silica from Grace Davison as the stationary phase and Geduran Si60 63-200 μm silica from Merck for the precolumn using an Interchim PuriFlash XS 420 automated flash chromatography system.

### Preparation of 1

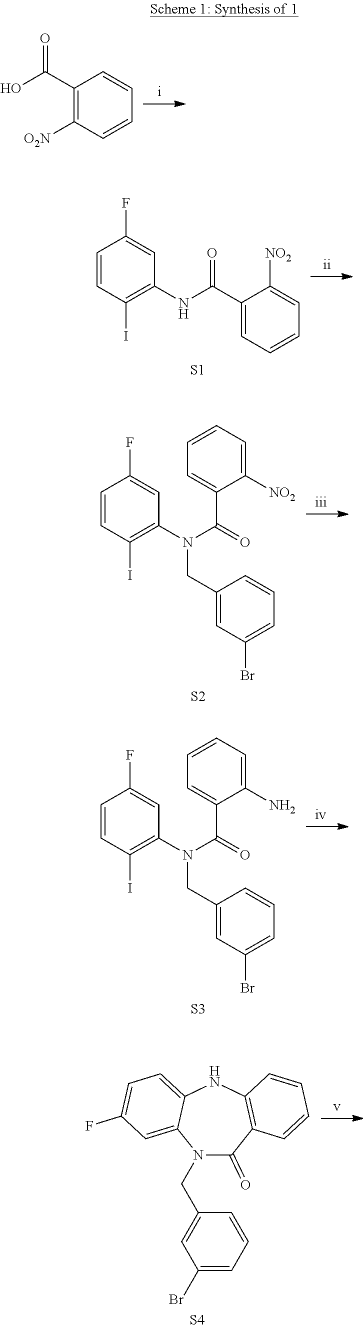

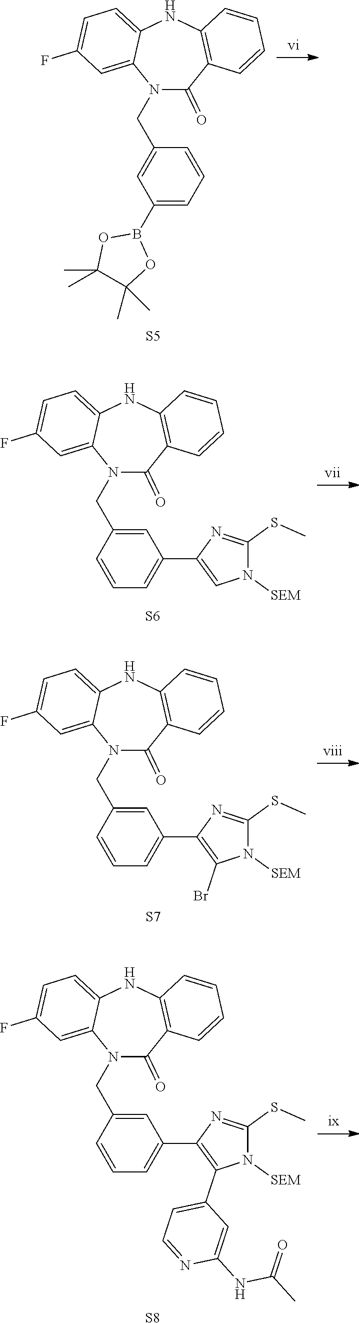

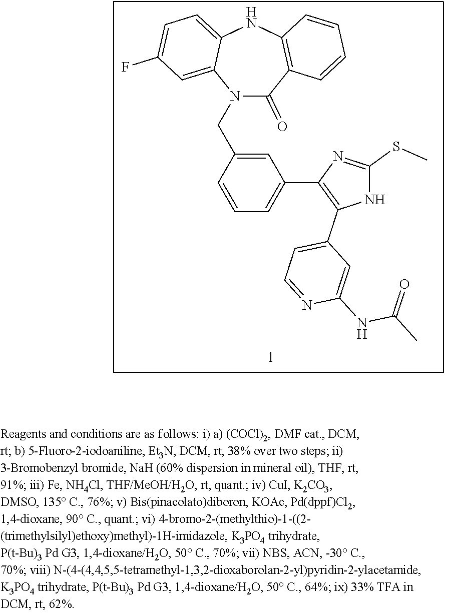 [SMILES: CSc1nc(-c2cccc(CN3C(=O)c4ccccc4Nc4ccc(F)cc43)c2)c(-c2ccnc(NC(C)=O)c2)[nH]1 ; "1"]

N-(5-Fluoro-2-iodophenyl)-2-nitrobenzamide (S1). To begin, 917 mg (5.48 mmol) 2-nitrobenzoic acid was dissolved in 20 ml DCM, and 0.4 ml of DMF was added to the mixture. 0.54 ml (6.33 mmol) oxalyl chloride was added drop wise under gas formation and the mixture was stirred for 1 h at ambient temperature, whereupon the excess of oxalyl chloride was removed in vacuo. At the same time, 1.00 g (4.22 mmol) 5-fluoro-2-iodoaniline was dissolved in 20 ml DCM, 1.8 ml (12.87 mmol) triethylamine was added, and the mixture was cooled down to 0° C. To the reaction mixture was slowly added the previously prepared acid chloride dissolved in 10 mL of DCM, whereupon the mixture was warmed to ambient temperature and stirred for 1 h. Brine was added to the reaction mixture and the aqueous layer was extracted with DCM. The combined organic layers were dried over Na₂SO₄, and solvents were removed in vacuo. Purification via flash chromatography (SiO₂; n-hexane/EtOAc 50:50) yielded 38% (611 mg, 1.58 mmol) of a light-yellow solid. ¹H NMR (400 MHz, DMSO) δ 10.39 (s, 1H), 8.19 (d, J=8.1 Hz, 1H), 7.97-7.89 (m, 2H), 7.84-7.75 (m, 2H), 7.40 (dd, J=10.1, 2.7 Hz, 1H), 7.02 (td, J=8.5, 2.9 Hz, 1H). ¹³C NMR (101 MHz, DMSO) δ 164.7, 162.0 (d, J=245.1 Hz), 146.4, 140.5 (d, J=10.3 Hz), 140.2 (d, J=8.7 Hz), 134.1, 132.1, 131.2, 129.0, 124.3, 115.5 (d, J=21.6 Hz), 114.2 (d, J=24.2 Hz), 90.1. TLC-MS (ESI+): calcd m/z 385.96 for C₁₃H₁₈FIN₂O₃, found 409.4 [M+Na]⁺.

N-(3-Bromobenzyl)-N-(5-fluoro-2-iodophenyl)-2-nitro benzamide (S2). To begin, 582 mg (1.51 mmol) of S1 was dissolved in 15 mL of THF under a nitrogen atmosphere, and the solution was cooled to 0° C. To the solution was added 66 mg (1.66 mmol) of a 60% dispersion in oil of sodium hydride portion wise. To the stirred reaction mixture was added 414 mg (1.66 mmol) of 3-bromobenzyl bromide portion wise and after the full addition, the mixture was warmed to ambient temperature and stirred there overnight until complete conversion. Brine was added, and the organic layer was separated. The aqueous phase was extracted with EtOAc twice, and the combined organic layers were dried over Na₂SO₄. After removal of solvents the product was purified via flash chromatography (SiO₂; n-hexane/EtOAc 50:50). Yield: 91% (765 mg, 1.38 mmol) of a yellowish solid. ¹H NMR (400 MHz, CDCl₃) δ 7.91-7.86 (m, 1H), 7.68-7.63 (m, 1H), 7.57 (d, J=7.6 Hz, 1H), 7.40 (t, J=7.5 Hz, 1H), 7.37-7.25 (m, 4H), 7.18-7.12 (m, 1H), 6.58-6.52 (m, 1H), 6.51-6.45 (m, 1H), 5.80 (d, J=14.4 Hz, 1H; CH₂), 4.17 (d, J=14.4 Hz, 1H; CH₂). ¹³C NMR (101 MHz, CDCl₃) δ 166.6, 162.6 (d, J=252.1 Hz), 145.9, 143.9 (d, J=9.7 Hz), 141.0 (d, J=8.5 Hz), 137.6, 133.8, 132.7, 132.1, 131.4, 130.4, 130.3, 128.5, 127.7, 124.6, 122.5, 118.9 (d, J=23.1 Hz), 118.0 (d, J=21.6 Hz), 92.8 (d, J=4.0 Hz), 50.9. TLC-MS (ESI+): calcd m/z 553.91 for C₂₀H₁₃BrFIN₂O₃, found 577.5/579.5 [M+Na]⁺.

2-Amino-N-(3-bromobenzyl)-N-(5-fluoro-2-iodophenyl)benzamide (S3). 700 mg (1.26 mmol) of S2, 352 mg (6.30 mmol) iron powder, and 674 mg (3.40 mmol) ammonium chloride were suspended in a mixture of THF/MeOH/H₂O (5:2:1, 12.5 mL). The resulting mixture was vigorously stirred at 50° C. for 1 h. Then, 72 μL of acetic acid was added and the mixture was stirred for another 1 h at 50° C. Thereupon, the reaction mixture was cooled to ambient temperature and filtered through a pad of Celite. To the filtrate, water was added and the aqueous phase was extracted with EtOAC, which was then washed three times with aqueous saturated NaHCO₃ solution. The organic layer was dried over Na₂SO₄, filtered and concentrated in vacuo. 666 mg (quant., 1.26 mmol) of a light-yellow solid were obtained. The product was used without further purification in the next step. TLC-MS (ESI+): calcd m/z 523.94 for C₂₀H₁₅BrFIN₂O, found 547.5/549.5 [M+Na]⁺.

10-(3-Bromobenzyl)-8-fluoro-5,10-dihydro-11H-dibenzo[b,e][1,4]diazepin-11-one (S4). 660 mg (1.26 mmol) of S3, 48 mg (0.25 mmol) copper (I) iodide, and 434 mg (3.14 mmol) of K₂CO₃ were suspended in 2 ml DMSO and the resulting reaction mixture was stirred at 135° C. for 2 hr. After cooling down to ambient temperature, the mixture was diluted with an excess of EtOAc and washed three times with water. The organic layer was dried over Na₂SO₄, filtered and concentrated in vacuo. The crude product was purified via flash chromatography (SiO₂; n-hexane/EtOAc 65:35) to yield 76% (380 mg, 0.96 mmol) of a light-yellow solid. ¹H NMR (400 MHz, DMSO) δ 7.96 (s, 1H), 7.65 (d, J=7.8 Hz, 1H), 7.52 (s, 1H), 7.41-7.35 (m, 2H), 7.32-7.23 (m, 3H), 7.16-7.11 (m, 1H), 7.09 (d, J=7.6 Hz, 1H), 7.02-6.97 (m, 1H), 6.94-6.89 (m, 1H), 5.29 (s, 2H). ¹³C NMR (101 MHz, DMSO) δ 167.9, 158.3 (d, J=238.9 Hz), 152.1, 141.8, 140.2, 134.2 (d, J=10.0 Hz), 132.8, 132.1, 130.5, 129.8, 129.3, 125.7, 124.2, 121.8, 121.7, 121.6 (d, J=9.3 Hz), 118.8, 112.7 (d, J=22.3 Hz), 110.9 (d, J=25.3 Hz), 50.9. TLC-MS (ESI+): calcd. m/z 396.03 for C₂₀H₁₄BrFN₂O, found 419.4/421.4 [M+Na]⁺.

8-Fluoro-10-(3-(4,4,5,5-tetramethyl-1,3,2-dioxaborolan-2-yl)benzyl)-5,10-dihydro-11H-dibenzo[b,e][1,4]diazepin-11-one (S5). To begin, 365 mg (0.92 mmol) of S4, 231 mg (0.91 mmol) bis(pinacolato)diboron and 271 mg (2.76 mmol) KOAc were suspended in 10 ml of 1,4-dioxane under an argon atmosphere. The suspension was degassed with three cycles of evacuation and backfilling with argon, followed by the addition of 34 mg of Pd(dppf)Cl₂ and another three cycles of degassing. The reaction mixture was heated to 90° C. and stirred overnight. The reaction mixture was then cooled down to ambient temperature and filtered through a pad of Celite. The filtrate was concentrated and 410 mg (quant., 0.92 mmol) of a brown oil was isolated and used without further purification in the next step. TLC-MS (ESI+): calcd. m/z 444.20 for C₂₆H₂₆BFN₂O₃, found 467.6 [M+Na]⁺.

8-Fluoro-10-(3-(2-(methylthio)-1-((2-(trimethylsilyl)ethoxy)methyl)-1H-imidazol-4-yl)benzyl)-5,10-dihydro-11H-dibenzo[b,e][1,4]diazepin-11-one (S6). To begin, 407 mg (0.92 mmol) of S5, 444 mg (1.37 mmol) of 4-bromo-2-(methylthio)-1-((2-(trimethylsilyl)ethoxy)methyl)-1H-imidazole, and 976 mg (3.66 mmol) of K₃PO₄ trihydrate were suspended in 9 mL of 1,4-dioxane and 2.5 mL of demineralized water. The solution was degassed with three cycles of evacuation and backfilling with argon, followed by the addition of 52 mg (10 mol %) of P(t-Bu)₃ Pd G3 and another three cycles of evacuation and argon backfilling. The reaction mixture was warmed to 50° C. and stirred overnight. After cooling to ambient temperature, brine was added to the mixture and the aqueous phase was extracted several times with EtOAc. The combined organic layers were dried over Na₂SO₄, filtered, and evaporated to dryness. The crude product was purified via flash chromatography (SiO₂; n-hexane/EtOAc 70:30) to obtain a yellow oil in a 70% yield (361 mg, 0.64 mmol). ¹H NMR (400 MHz, DMSO) δ 7.88 (s, 1H), 7.77 (s, 1H), 7.72 (s, 1H), 7.68-7.65 (m, 1H), 7.59-7.55 (m, 1H), 7.39-7.35 (m, 1H), 7.29-7.27 (m, 1H), 7.25 (dd, J=7.5, 3.1 Hz, 1H), 7.17-7.11 (m, 2H), 7.10-7.07 (m, 1H), 7.02-6.98 (m, 1H), 6.91-6.86 (m, 1H), 5.30 (s, 2H), 5.28 (s, 2H), 3.55-3.50 (m, 2H), 2.58 (s, 3H), 0.88-0.84 (m, 2H), −0.04 (s, 9H). ¹³C NMR (101 MHz, DMSO) δ 167.9, 158.2 (d, J=238.6 Hz), 152.1, 143.1, 141.7 (d, J=2.3 Hz), 140.3, 137.6, 134.5 (d, J=10.1 Hz), 133.9, 132.6, 132.1, 128.6, 124.7, 124.4, 122.8, 122.6, 121.7, 121.5 (d, J=9.1 Hz), 118.8, 118.4, 112.4 (d, J=22.4 Hz), 110.7 (d, J=25.1 Hz), 73.5, 65.5, 51.7, 17.1, 15.8, −1.4. (With residues of pinacol). TLC-MS (ESI+): calcd. m/z 560.21 for C₃₀H₃₃FN₄O₂SSi, found 584.0 [M+Na]⁺.

10-(3-(5-Bromo-2-(methylthio)-1-((2-(trimethylsilyl)ethoxy)methyl)-1H-imidazol-4-yl)benzyl)-8-fluoro-5,10-dihydro-11H-dibenzo[b,e][1,4]diazepin-11-one (S7). To begin, 323 mg (0.58 mmol) of S6 was dissolved in 10 mL of acetonitrile (ACN) under an argon atmosphere and the solution was cooled to −30° C. Then, 0.92 mg (0.52 mmol) of N-bromosuccinimide dissolved in 10 mL of ACN was added dropwise to the solution, while the temperature was maintained at −30° C. The reaction mixture was stirred for 1 h at −30° C. and then slowly warmed up to ambient temperature. The reaction mixture was quenched by the addition of an aqueous saturated Na₂SO₃ solution, and the aqueous phase was extracted several times with EtOAc. The organic layers were dried over Na₂SO₄, filtered, and evaporated to dryness. The crude product was purified via flash chromatography (SiO₂; n-hexane/EtOAc 65:35) to obtain a yellowish solid in a 70% yield (260 mg, 0.41 mmol). ¹H NMR (400 MHz, DMSO) δ 7.87 (s, 1H), 7.85 (s, 1H), 7.75 (d, J=7.6 Hz, 1H), 7.67 (d, J=7.7 Hz, 1H), 7.39-7.34 (m, 2H), 7.29-7.26 (m, 1H), 7.24 (s, 1H), 7.14-7.10 (m, 1H), 7.09-7.06 (m, 1H), 7.01-6.96 (m, 1H), 6.92-6.87 (m, 1H), 5.31 (s, 2H), 5.30 (s, 2H), 3.58 (t, J=7.8 Hz, 2H), 2.60 (s, 3H), 0.87 (t, J=7.9 Hz, 2H), −0.04 (s, 9H). ¹³C NMR (101 MHz, DMSO) δ 167.8, 158.2 (d, J=238.6 Hz), 152.1, 144.9, 141.6, 141.5, 137.8, 137.6, 134.55 (d, J=9.9 Hz), 132.7 (d, J=8.6 Hz), 132.1, 128.5, 125.7, 124.7, 124.5, 124.2, 121.6, 121.5, 118.7, 112.4 (d, J=22.6 Hz), 110.6 (d, J=25.6 Hz), 100.6, 73.6, 65.9, 51.8, 17.2, 15.5, −1.4. TLC-MS (ESI+): calcd. m/z 638.12 for C₃₀H₃₂BrFN₄O₂SSi, found 660.6/662.5 [M+Na]⁺.

N-(4-(4-(3-((8-Fluoro-11-oxo-5,11-dihydro-10H-dibenzo[b,e][1,4]diazepin-10-yl)methyl)phenyl)-2-(methylthio)-1-((2-(trimethylsilyl)ethoxy)methyl)-1H-imidazol-5-yl)pyridin-2-yl) acetamide (S8). To begin, 260 mg (0.41 mmol) of S7, 197 mg (0.61 mmol) of N-(4-(4,4,5,5-tetramethyl-1,3,2-dioxaborolan-2-yl)pyridin-2-yl) acetamide, and 433 mg (1.63 mmol) of K₃PO₄ trihydrate were suspended in 4 mL of 1,4-dioxane and 1 mL of demineralized water. The solution was degassed with three cycles of evacuation and backfilling with argon, followed by the addition of 23 mg (10 mol %) of P(t-Bu)₃ Pd G3 and another three cycles of evacuation and argon backfilling. The reaction mixture was warmed to 50° C. and stirred overnight. After cooling down to ambient temperature, brine was added to the mixture and the aqueous phase was extracted several times with EtOAc. Combined organic layers were dried over Na₂SO₄, filtered, and evaporated to dryness. The crude product was purified via flash chromatography (SiO₂; n-hexane/EtOAc 50:50) to obtain a yellow solid in a 64% yield (180 mg, 0.26 mmol). ¹H NMR (400 MHz, DMSO) δ 10.52 (s, 1H), 8.11 (d, J=5.0 Hz, 1H), 8.08 (s, 1H), 7.82 (s, 1H), 7.60 (d, J=7.2 Hz, 1H), 7.46 (s, 1H), 7.37 (t, J=7.0 Hz, 1H), 7.23-7.18 (m, 2H), 7.16-7.13 (m, 1H), 7.13-7.09 (m, 1H), 7.08-7.03 (m, 2H), 7.02-6.98 (m, 1H), 6.97-6.95 (m, 1H), 6.89 (td, J=8.5, 2.6 Hz, 1H), 5.15 (s, 2H), 5.08 (s, 2H), 3.37-3.33 (m, 2H), 2.66 (s, 3H), 2.05 (s, 3H), 0.79-0.75 (m, 2H), −0.09 (s, 9H). ¹³C NMR (101 MHz, DMSO) δ 169.2, 167.7, 158.1 (d, J=238.6 Hz), 152.6, 151.9, 148.3, 144.9, 141.28, 141.3, 139.5, 138.1, 137.5, 134.6 (d, J=9.9 Hz), 133.7, 132.6, 132.2, 128.3, 127.9, 125.0, 125.0, 124.3, 121.6, 121.5 (d, J=9.1 Hz), 120.4, 118.7, 114.2, 112.4 (d, J=22.1 Hz), 110.3 (d, J=25.3 Hz), 72.5, 65.5, 52.1, 23.8, 17.1, 15.5, −1.5. TLC-MS (ESI+): calcd. m/z 694.26 for C₃₇H₃₉FN₆O₃SSi, found 717.0 [M+Na]⁺.

N-(4-(4-(3-((8-Fluoro-11-oxo-5,11-dihydro-10H-dibenzo[b,e][1,4]diazepin-10-yl)methyl)phenyl)-2-(methylthio)-1H-imidazol-5-yl)pyridin-2-yl) acetamide (1). 40 mg (0.06 mmol) of S8 was dissolved in 3 ml of a mixture of TFA/DCM 33% v/v. The solution was stirred overnight at ambient temperature. After quenching the reaction mixture with a saturated aqueous NaHCO₃ solution, the aqueous layer was extracted several times with EtOAc. The combined organic layers were dried over Na₂SO₄, filtered, and the solvents removed in vacuo. The crude product was purified via flash chromatography (SiO₂; n-hexane/EtOAc/MeOH 10:85:5) to give the pure product as a white solid in a 62% yield (20 mg, 0.04 mmol). As mixture of tautomers: ¹H NMR (400 MHz, DMSO) δ 12.82-12.62 (m, 1H), 10.46-10.23 (m, 1H), 8.35-7.93 (m, 2H), 7.84 (s, 1H), 7.57 (d, J=7.6 Hz, 1H), 7.49-7.42 (m, 1H), 7.41-7.23 (m, 4H), 7.18 (d, J=9.2 Hz, 1H), 7.12-7.02 (m, 2H), 6.96 (t, J=7.5 Hz, 1H), 6.93-6.83 (m, 2H), 5.27 (s, 2H), 2.61 (s, 3H), 2.05 (s, 3H). ¹³C NMR (101 MHz, DMSO) δ 168.9, 167.8, 158.2 (d, J=238.6 Hz), 152.4, 152.0, 147.3, 143.7, 142.1, 141.5, 138.1, 134.6 (d, J=10.2 Hz), 134.4, 132.6, 132.1, 131.0, 130.3, 128.9, 127.0, 126.9, 126.3, 124.3, 121.7, 121.5 (d, J=9.3 Hz), 118.8, 116.2, 112.5 (d, J=21.8 Hz), 110.6 (d, J=24.8 Hz), 110.4, 51.9, 23.8, 15.1. HRMS (ESI): exact mass calcd, for C₃₁H₂₅FN₆O₂S [M+H]⁺: 565.18165, found: 565.18227.

### Preparation of 2-ethyl-imidazole precursors

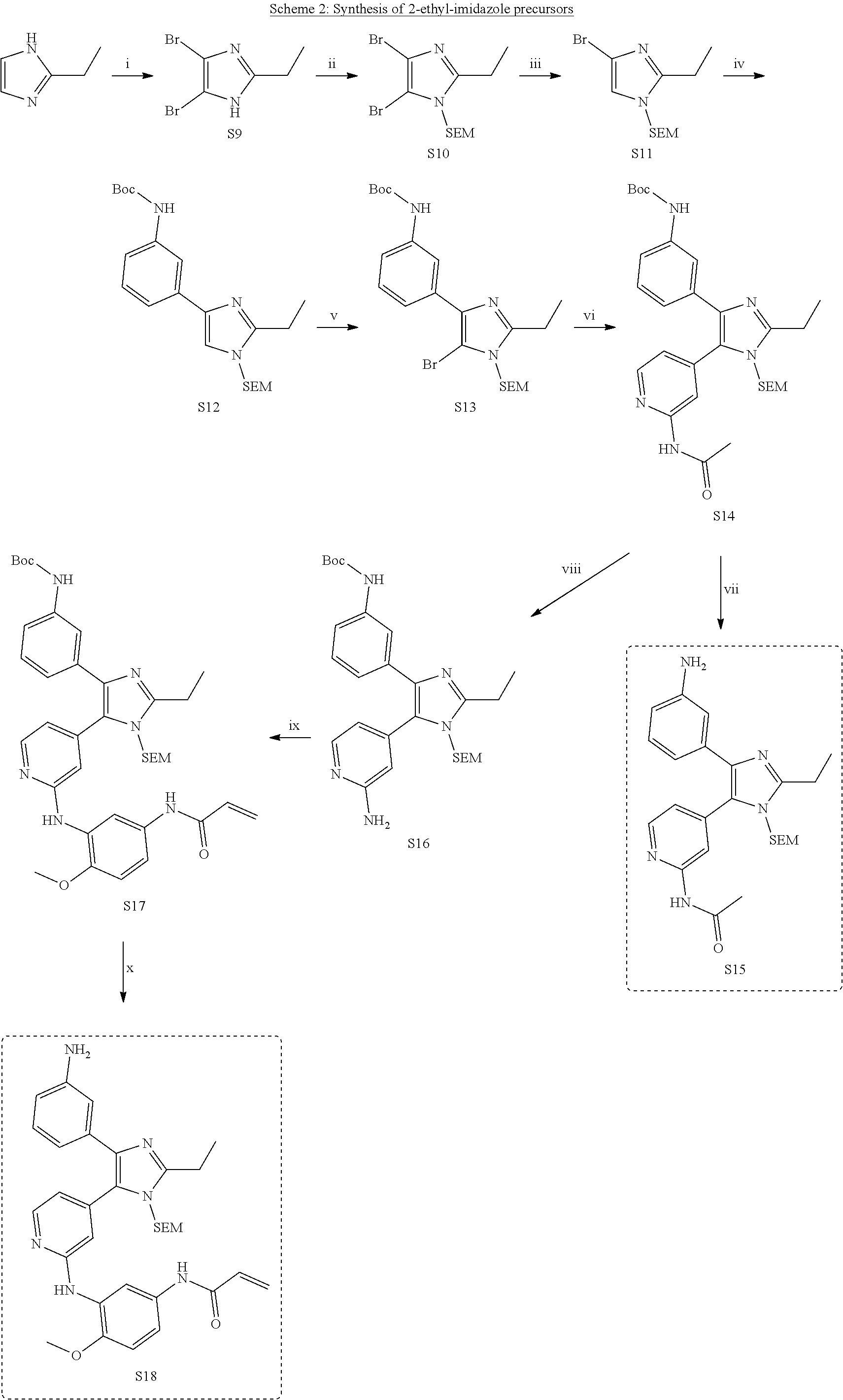

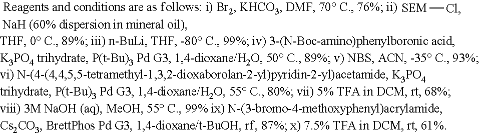

4,5-Dibromo-2-ethyl-1H-imidazole (S9). To begin, 10.04 g (96.13 mmol) of 2-ethylimidazole was dissolved in 50 ml of DMF and 31.37 g (313.3 mmol) KHCO₃ was added. The suspension was cooled down to 0° C. and 11 ml (214.1 mmol) of bromine was added dropwise under exothermic reaction. After complete addition, the reaction mixture was warmed up to 70° C. and stirred overnight. After cooling down to 0° C., 150 ml of iced water was added, and the mixture was stirred for several minutes under formation of a yellow precipitate. The solid was filtered, washed with water, and dried in the oven. Yield: 20.16 g (76%, 79.41 mmol) as off-white solid. ¹H NMR (400 MHz, CDCl₃) δ 11.90 (s, 1H), 2.79 (q, J=7.5 Hz, 2H), 1.29 (t, J=7.5 Hz, 3H). ¹³C NMR (101 MHz, CDCl₃) δ 151.6, 106.8, 22.4, 12.7.

4,5-Dibromo-2-ethyl-1-((2-(trimethylsilyl)ethoxy)methyl)-1H-imidazole (S10). To begin, 11.24 g (44.26 mmol) of S9 was dissolved in 60 mL of THF under a nitrogen atmosphere, and the solution was cooled to 0° C. To the solution was added 2.04 g (50.9 mmol) of a 60% dispersion in oil of sodium hydride portion wise while maintaining a temperature under 0° C. To the stirred reaction mixture was added 8.38 mL (47.4 mmol) of SEM-Cl dissolved in 30 ml THF dropwise while the temperature was maintained under 10° C. After the full addition, the mixture was warmed up to ambient temperature and stirred overnight. To the mixture was added brine and the aqueous phase was extracted several times with DCM. The combined organic layers were dried over Na₂SO₄, filtered, and the solvents removed in vacuo. The crude product was purified via flash chromatography (SiO₂; isocratic, n-hexane/EtOAc 95:5) to give the product as colorless oil in 89% yield (15.12 g, 39.36 mmol). ¹H NMR (400 MHz, DMSO) δ 5.29 (s, 1H), 3.56-3.50 (m, 1H), 2.73 (q, J=7.5 Hz, 1H), 1.19 (t, J=7.5 Hz, 2H), 0.88-0.83 (m, 1H), −0.04 (s, 4H). ¹³C NMR (101 MHz, DMSO) δ 152.2, 115.9, 103.9, 74.4, 66.5, 21.1, 18.1, 12.3, −0.5.

4-Bromo-2-ethyl-1-((2-(trimethylsilyl)ethoxy)methyl)-1H-imidazole (S11). To begin, 10.0 g (26.0 mmol) of S10 was dissolved in 60 mL of THF under an argon atmosphere, and the solution was cooled to −80° C. To the solution was added 10.4 mL (26.0 mmol) of 2.5 M n-BuLi in n-hexane solution dropwise via syringe while the temperature was maintained at −80° C. After full addition, the reaction mixture was stirred for 15 minutes, whereupon MeOH was added, and the mixture was warmed up to ambient temperature. To the mixture was added brine, and the organic layer was separated. The aqueous phase was extracted several times with EtOAc. The combined organic layers were dried over Na₂SO₄, and the solvent was removed in vacuo. The product was obtained as a yellow oil (99%, 7.90 g, 25.9 mmol) and was used without further purification in the next step. ¹H NMR (400 MHz, DMSO) δ 7.32 (s, 1H), 5.24 (s, 2H), 3.50-3.45 (m, 2H), 2.65 (q, J=7.5 Hz, 2H), 1.18 (t, J=7.5 Hz, 3H), 0.86-0.81 (m, 2H), −0.04 (s, 9H). ¹³C NMR (101 MHz, DMSO) δ 149.8, 119.4, 112.2, 74.1, 65.3, 19.3, 17.1, 11.9, −1.4.

tert-Butyl (3-(2-ethyl-1-((2-(trimethylsilyl)ethoxy)methyl)-1H-imidazol-4-yl)phenyl)carbamate (S12). To begin, 2.50 g (8.19 mmol) of S11, 2.14 g (9.01 mmol) of 3-(N-Boc-amino)phenylboronic acid, and 8.72 g (32.8 mmol) of K₃PO₄ trihydrate were dissolved in 55 mL of 1,4-dioxane and 15 mL of demineralized water. The solution was degassed with three cycles of evacuation and backfilling with argon. To the solution was added 70 mg (1.5 mol %) of P(t-Bu)₃ Pd G3, and another three cycles of evacuation and argon backfilling were carried out. The reaction mixture was warmed to 50° C. and stirred overnight. After cooling to ambient temperature, brine was added and the aqueous phase was extracted several times with EtOAc. The combined organic layers were dried over Na₂SO₄, filtered, and evaporated to dryness. The crude product was purified via flash chromatography (SiO₂; n-hexane/EtOAc 70:30) to obtain a yellow solid in 89% yield (3.05 g, 7.30 mmol). ¹H NMR (400 MHz, DMSO) δ 9.29 (s, 1H), 7.94 (s, 1H), 7.53 (s, 1H), 7.33-7.29 (m, 1H), 7.23-7.16 (m, 2H), 5.29 (s, 2H), 3.55-3.49 (m, 2H), 2.71 (q, J=7.5 Hz, 2H), 1.48 (s, 9H), 1.25 (t, J=7.5 Hz, 3H), 0.88-0.83 (m, 2H), −0.04 (s, 9H). ¹³C NMR (101 MHz, DMSO) δ 152.8, 149.7, 139.7, 138.3, 134.9, 128.6, 118.2, 116.5, 116.2, 114.1, 78.8, 74.2, 65.1, 28.1, 19.4, 17.2, 12.4, −1.7. TLC-MS (ESI+): calcd. m/z 417.24 for C₂₂H₃₅N₃O₃Si, found 418.5 [M+H]⁺.

tert-Butyl (3-(5-bromo-2-ethyl-1-((2-(trimethylsilyl)ethoxy)methyl)-1H-imidazol-4-yl)phenyl)carbamate (S13). To begin, 2.90 g (6.94 mmol) of S12 was dissolved in 50 mL of ACN under an argon atmosphere and the solution was cooled to −30° C. Then, 1.24 g (6.94 mmol) of N-bromosuccinimide dissolved in 20 mL of ACN was added dropwise to the solution, while the temperature was maintained at −30° C. The reaction mixture was stirred for 1 h at −30° C. and then slowly warmed to ambient temperature. The reaction mixture was quenched by the addition of an aqueous saturated Na₂SO₃ solution, and the aqueous phase was extracted several times with EtOAc. The organic layers were dried over Na₂SO₄, filtered, and evaporated to dryness. The crude product was purified via flash chromatography (SiO₂; n-hexane/EtOAc 70:30) to obtain a light-yellow oil in 93% yield (3.19 g, 6.42 mmol). ¹H NMR (400 MHz, DMSO) δ 9.38 (s, 1H), 8.04 (s, 1H), 7.49 (d, J=7.7 Hz, 1H), 7.38 (d, J=7.8 Hz, 1H), 7.27 (t, J=7.9 Hz, 1H), 5.33 (s, 2H), 3.58 (t, J=8.0 Hz, 2H), 2.78 (q, J=7.4 Hz, 2H), 1.48 (s, 9H), 1.26 (t, J=7.5 Hz, 3H), 0.88 (t, J=8.0 Hz, 2H), −0.03 (s, 9H). ¹³C NMR (101 MHz, DMSO) δ 152.8, 150.9, 139.6, 135.9, 133.6, 128.4, 120.0, 116.9, 116.3, 99.2, 78.9, 72.7, 65.4, 28.1, 20.2, 17.3, 11.8, −1.4. TLC-MS (ESI+): calcd. m/z 495.16 for C₂₂H₃₄BrN₃O₃Si, found 517.7/519.7 [M+Na]⁺.

tert-Butyl (3-(5-(2-acetamidopyridin-4-yl)-2-ethyl-1-((2-(trimethylsilyl)ethoxy)methyl)-1H-imidazol-4-yl)phenyl)carbamate (S14). To begin, 2.70 g (5.44 mmol) of S13, 2.00 g (7.61 mmol) of N-(4-(4,4,5,5-tetramethyl-1,3,2-dioxaborolan-2-yl)pyridin-2-yl) acetamide, and 5.79 g (21.8 mmol) of K₃PO₄ trihydrate were dissolved in 55 mL of 1,4-dioxane and 15 mL of demineralized water. The solution was degassed with three cycles of evacuation and backfilling with argon. To the solution was added 50 mg (1.5 mol %) of P(t-Bu)₃ Pd G3, and another three cycles of evacuation and argon backfilling were carried out. The reaction mixture was warmed to 55° C. and stirred overnight. After cooling to ambient temperature, brine was added and the aqueous phase was extracted several times with EtOAc. The combined organic layers were dried over Na₂SO₄, filtered, and evaporated to dryness. The crude product was purified via flash chromatography (SiO₂; n-hexane/EtOAc 40:60) to obtain an off-white solid in 80% yield (2.40 g, 4.35 mmol). ¹H NMR (400 MHz, DMSO) δ 10.61 (s, 1H), 9.24 (s, 1H), 8.37-8.32 (m, 1H), 8.10 (s, 1H), 7.71-7.65 (m, 1H), 7.25 (d, J=7.7 Hz, 1H), 7.05 (t, J=7.9 Hz, 1H), 6.98 (dd, J=5.1, 1.5 Hz, 1H), 6.87-6.82 (m, 1H), 5.10 (s, 2H), 3.33-3.29 (m, 2H), 2.80 (q, J=7.5 Hz, 2H), 2.07 (s, 3H), 1.45 (s, 9H), 1.32 (t, J=7.5 Hz, 3H), 0.79-0.74 (m, 2H), −0.09 (s, 9H). ¹³C NMR (101 MHz, DMSO) δ 169.2, 152.7, 152.7, 150.5, 148.4, 140.3, 139.5, 136.5, 134.6, 128.2, 126.0, 120.8, 120.5, 116.9, 116.5, 114.6, 78.8, 71.8, 65.1, 28.1, 23.88, 19.8, 17.2, 12.1, −1.5. (With residues of pinacol). TLC-MS (ESI+): calcd. m/z 551.29 for C₂₉H₄₁N₅O₄Si, found 574.1 [M+Na]⁺.

N-(4-(4-(3-Aminophenyl)-2-ethyl-1-((2-(trimethylsilyl)ethoxy)methyl)-1H-imidazol-5-yl)pyridin-2-yl) acetamide (S15). 900 mg (1.63 mmol) of S14 was dissolved in 32 ml of a mixture of TFA/DCM 5% v/v. The solution was stirred overnight at ambient temperature. The reaction mixture was concentrated in vacuo and the residue was quenched with a saturated aqueous NaHCO₃ solution, whereupon the aqueous layer was extracted several times with EtOAc. The combined organic layers were dried over Na₂SO₄, filtered, and the solvents removed in vacuo. The crude product was purified via flash chromatography (SiO₂; n-hexane/EtOAc/MeOH 10:80:5) to give the pure product as a white solid in a 68% yield (500 mg, 1.11 mmol). ¹H NMR (400 MHz, DMSO) δ 10.61 (s, 1H), 8.33 (d, J=5.0 Hz, 1H), 8.10 (s, 1H), 6.98 (dd, J=5.1, 1.4 Hz, 1H), 6.87-6.83 (m, 1H), 6.80 (t, J=7.8 Hz, 1H), 6.39-6.32 (m, 2H), 5.08 (s, 2H), 4.97 (s, 2H), 3.33-3.27 (m, 2H), 2.78 (q, J=7.5 Hz, 2H), 2.08 (s, 3H), 1.31 (t, J=7.5 Hz, 3H), 0.79-0.73 (m, 2H), −0.09 (s, 9H). ¹³C NMR (101 MHz, DMSO) δ 308.7, 292.1, 289.7, 287.9, 287.7, 280.2, 276.7, 274.2, 267.9, 264.9, 260.5, 254.1, 252.2, 251.9, 211.2, 204.5, 163.4, 159.2, 156.66, 151.6, 138.0. TLC-MS (ESI+): calcd. m/z 451.24 for C₂₄H₃₃N₅O₂Si, found 473.9 [M+Na]⁺.

tert-Butyl (3-(5-(2-aminopyridin-4-yl)-2-ethyl-1-((2-(trimethylsilyl)ethoxy)methyl)-1H-imidazol-4-yl)phenyl)carbamate (S16). 840 mg (1.52 mmol) of S14 was dissolved in 25 mL of MeOH and 5 mL of a 3 N NaOH solution was added. The reaction mixture was stirred at 55° C. for 6 h. After cooling down to ambient temperature, solvents were concentrated in vacuo. The oily residue was dissolved in small amounts of MeOH and a slow addition of iced water precipitated a white solid, which was collected by filtration and dried in the oven to obtain the product in 99% yield (770 mg, 1.51 mmol). ¹H NMR (400 MHz, DMSO) δ 9.26 (s, 1H), 7.97 (d, J=5.1 Hz, 1H), 7.75 (s, 1H), 7.25 (d, J=7.6 Hz, 1H), 7.05 (t, J=7.9 Hz, 1H), 6.83 (d, J=7.7 Hz, 1H), 6.42 (d, J=5.1 Hz, 1H), 6.35 (s, 1H), 6.02 (s, 2H), 5.10 (s, 2H), 3.35-3.29 (m, 2H), 2.78 (q, J=7.4 Hz, 2H), 1.46 (s, 9H), 1.31 (t, J=7.5 Hz, 3H), 0.80-0.74 (m, 2H), −0.07 (s, 9H). ¹³C NMR (101 MHz, DMSO) δ 160.2, 152.8, 150.0, 148.4, 139.5, 139.3, 135.7, 134.9, 128.1, 126.8, 120.5, 117.0, 116.4, 113.4, 109.4, 78.8, 71.7, 65.1, 28.1, 19.8, 17.2, 12.1, −1.4. TLC-MS (ESI+): calcd. m/z 509.28 for C₂₇H₃₉N₅O₃Si, found 532.1 [M+Na].

tert-Butyl (3-(5-(2-((5-acrylamido-2-methoxyphenyl)amino)pyridin-4-yl)-2-ethyl-1-((2-(trimethylsilyl)ethoxy)methyl)-1H-imidazol-4-yl)phenyl)carbamate (S17). To begin, 500 mg (0.98 mmol) of S16, 327 mg (1.28 mmol) of N-(3-bromo-4-methoxyphenyl) acrylamide, and 415 mg of Cs₂CO₃ (1.28 mmol) in 10 mL of a mixture of 1,4-dioxane/t-BuOH (4+1) was degassed three times by evacuating and backfilling with argon under stirring. The mixture was added 44 mg of BrettPhos Pd G3 (5 mol %), and another three cycles of evacuation and argon backfilling were carried out. The solution was stirred under reflux for 4 h. After cooling to ambient temperature, Celite was added to the mixture, and solvents were removed in vacuo. Purification via flash chromatography (SiO₂; n-hexane/EtOAc 30:70) yielded 87% (585 mg, 0.85 mmol) of a yellow solid. ¹H NMR (400 MHz, DMSO) δ 9.96 (s, 1H), 9.23 (s, 1H), 8.42 (d, J=2.5 Hz, 1H), 8.20 (d, J=5.2 Hz, 1H), 8.18 (s, 1H), 7.75 (t, J=1.5 Hz, 1H), 7.41 (dd, J=8.8, 2.5 Hz, 1H), 7.25 (dd, J=8.1, 0.8 Hz, 1H), 7.07 (t, J=7.9 Hz, 1H), 6.99 (s, 1H), 6.94 (d, J=8.9 Hz, 1H), 6.90-6.87 (m, 1H), 6.65 (dd, J=5.2, 1.2 Hz, 1H), 6.45 (dd, J=17.0, 10.1 Hz, 1H), 6.22 (dd, J=17.0, 2.1 Hz, 1H), 5.69 (dd, J=10.1, 2.0 Hz, 1H), 5.14 (s, 2H), 3.78 (s, 3H), 3.34-3.29 (m, 2H), 2.80 (q, J=7.5 Hz, 2H), 1.43 (s, 9H), 1.32 (t, J=7.5 Hz, 3H), 0.76-0.72 (m, 2H), −0.10 (s, 9H). ¹³C NMR (101 MHz, DMSO) δ 162.8, 156.3, 152.8, 150.3, 147.7, 145.4, 139.5, 139.5, 136.2, 134.8, 132.2, 131.9, 129.8, 128.2, 126.4, 126.1, 120.6, 117.1, 116.5, 116.1, 113.1, 112.5, 111.8, 110.8, 78.9, 71.8, 65.2, 55.9, 28.1, 19.8, 17.2, 12.2, −1.5. TLC-MS (ESI+): calcd.m/z 684.35 for C₃₇H₄₈N₆O₅Si, found 708.3 [M+Na]⁺.

N-(3-((4-(4-(3-Aminophenyl)-2-ethyl-1-((2-(trimethylsilyl)ethoxy)methyl)-1H-imidazol-5-yl)pyridin-2-yl)amino)-4-methoxyphenyl) acrylamide (S18). 570 mg (0.84 mmol) of S17 was dissolved in 10.5 ml of a mixture of TFA/DCM 7.5% v/v. The solution was stirred for 24 h at ambient temperature. The reaction mixture was concentrated in vacuo and the residue was quenched with a saturated aqueous NaHCO₃ solution, whereupon the aqueous layer was extracted several times with EtOAc. The combined organic layers were dried over Na₂SO₄, filtered, and the solvents removed in vacuo. The crude product was purified via flash chromatography (SiO₂; n-hexane/EtOAc/MeOH 10:80:10) to give the product as a yellow solid in a 61% yield (300 mg, 0.51 mmol). ¹H NMR (400 MHz, DMSO) δ 9.97 (s, 1H), 8.41 (d, J=2.5 Hz, 1H), 8.21-8.17 (m, 2H), 7.43 (dd, J=8.8, 2.5 Hz, 1H), 7.00 (s, 1H), 6.95 (d, J=8.9 Hz, 1H), 6.92-6.90 (m, 1H), 6.83 (t, J=7.8 Hz, 1H), 6.66 (dd, J=5.2, 1.2 Hz, 1H), 6.49-6.42 (m, 2H), 6.37 (ddd, J=8.0, 2.2, 0.8 Hz, 1H), 6.22 (dd, J=17.0, 2.1 Hz, 1H), 5.70 (dd, J=10.1, 2.1 Hz, 1H), 5.12 (s, 2H), 4.96 (s, 2H), 3.79 (s, 3H), 3.31-3.29 (m, 2H), 2.78 (q, J=7.5 Hz, 2H), 1.31 (t, J=7.5 Hz, 3H), 0.77-0.72 (m, 2H), −0.09 (s, 9H). ¹³C NMR (101 MHz, DMSO) δ 162.7, 156.2, 149.9, 148.4, 147.4, 145.4, 139.9, 136.7, 134.9, 132.2, 131.9, 129.8, 128.3, 125.9, 125.8, 116.2, 114.7, 112.9, 112.7, 112.5, 112.3, 111.8, 110.8, 71.7, 65.1, 55.8, 19.7, 17.2, 12.1, −1.5. TLC-MS (ESI+): calcd.m/z 584.80 for C₃₂H₄₀N₆O₃Si, found 607.8 [M+Na]⁺.

### Preparation of Dibenzodiazepinone-Carboxylic Acid Precursor

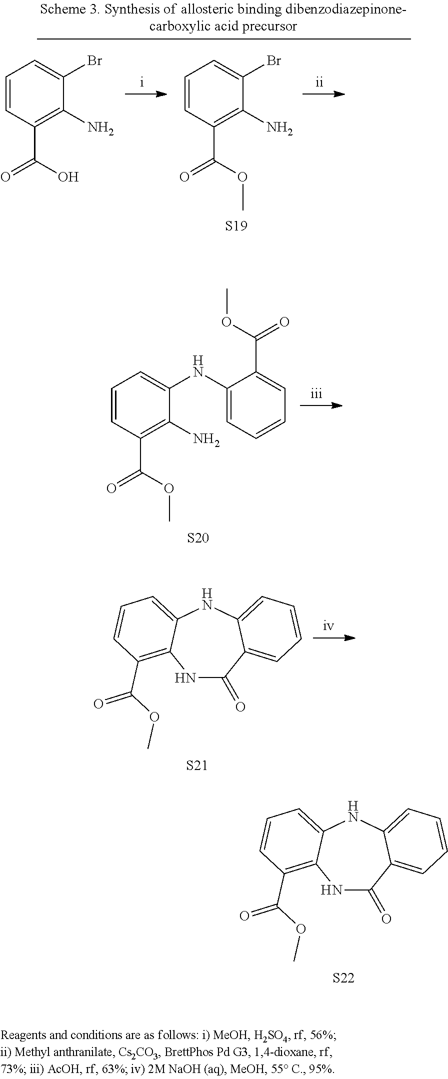

Methyl 2-amino-3-bromobenzoate (S19). 1.08 g (5.00 mmol) 2-amino-3-bromo-benzoic acid was dissolved in 10 ml of MeOH, 2 ml of concentrated H₂SO₄ was added and the reaction mixture was refluxed for 3 days. After cooling down to 0° C. the reaction mixture was quenched with saturated NaHCO₃ solution under precipitation of a brown solid, which was collected by filtration. The solid was thoroughly washed with n-hexane and the filtrate was concentrated in vacuo to give a light-yellow solid in 56% yield (650 mg, 2.83 mmol). ¹H NMR (400 MHz, DMSO) δ 7.78 (dd, J=8.0, 1.4 Hz, 1H), 7.67 (dd, J=7.7, 1.4 Hz, 1H), 6.70 (s, 2H), 6.55 (t, J=7.9 Hz, 1H), 3.82 (s, 3H). ¹³C NMR (101 MHz, DMSO) δ 167.3, 147.6, 137.5, 130.6, 116.1, 110.9, 109.7, 51.9. TLC-MS (ESI−): calcd. m/z 228.97 for C₈H₈BrNO₂, found 213.7/215.6 [M-CH₄]⁻.

Methyl 2-amino-3-((2-(methoxycarbonyl)phenyl)amino)benzoate (S20). To begin, 370 mg (1.61 mmol) of S19, 218 μL (1.69 mmol) of methyl anthranilate, and 1048 mg of Cs₂CO₃ (3.22 mmol) in 10 mL of 1,4-dioxane were degassed three times by evacuating and backfilling with argon under stirring. The mixture was added 36 mg of BrettPhos Pd G3 (2.5 mol %), and another three cycles of evacuation and argon backfilling were carried out. The solution was stirred at 100° C. overnight. After cooling to ambient temperature, demineralized water was added, and the aqueous phase was extracted several times with EtOAc. The combined organic layers were dried over Na₂SO₄, filtered, and the solvents removed in vacuo. The crude product was obtained in 73% yield (350 mg, 1.17 mmol) and used without further purification in the next step. ¹H NMR (400 MHz, DMSO) δ 8.78 (s, 1H), 7.88 (dd, J=8.0, 1.4 Hz, 1H), 7.71 (dd, J=8.1, 1.1 Hz, 1H), 7.34-7.29 (m, 2H), 6.75-6.70 (m, 1H), 6.65-6.60 (m, 1H), 6.51 (d, J=8.4 Hz, 1H), 6.47 (s, 2H), 3.86 (s, 3H), 3.82 (s, 3H). ¹³C NMR (101 MHz, DMSO) δ 168.2, 167.9, 148.8, 147.6, 134.5, 131.8, 131.1, 128.6, 126.5, 116.6, 114.9, 113.5, 111.2, 110.4, 51.8, 51.7. TLC-MS (ESI+): calcd. m/z 300.11 for C₁₆H₁₆N₂O₄, found 323.1 [M+Na]⁺.

Methyl 11-oxo-10,11-dihydro-5H-dibenzo[b,e][1,4]diazepine-9-carboxylate (S21). To 300 mg (1.00 mmol) of S20 was added 10 ml of glacial acetic acid and the reaction mixture was refluxed for 72 h. After cooling down to 0° C., the reaction mixture was neutralized by the careful addition of an aqueous 3 M NaOH solution under precipitation of a green solid, which was collected by filtration washed with iced water and dried in the oven. The product was obtained as green solid in 63% yield (170 mg, 0.63 mmol). ¹H NMR (400 MHz, DMSO) δ 10.24-9.89 (m, 1H), 8.09 (s, 1H), 7.69 (d, J=5.2 Hz, 1H), 7.53 (d, J=5.6 Hz, 1H), 7.39 (s, 1H), 7.29 (d, J=5.4 Hz, 1H), 7.17-7.01 (m, 2H), 6.94 (s, 1H), 3.86 (s, 3H). ¹³C NMR (101 MHz, DMSO) δ 167.6, 167.3, 150.2, 141.1, 133.8, 132.0, 131.4, 125.1, 124.6, 124.2, 122.3, 121.3, 119.3, 119.3, 52.6. TLC-MS (ESI+): calcd. m/z 268.08 for C₁₅H₁₂N₂O₃, found 291.1 [M+Na]⁺.

11-Oxo-10,11-dihydro-5H-dibenzo[b,e][1,4]diazepine-9-carboxylic acid (S22). 150 mg (0.56 mmol) of S21 was dissolved in 6 ml of MeOH and 0.5 ml of an aqueous 2 N NaOH solution was added. The reaction mixture was stirred overnight at ambient temperature until complete conversion. The mixture was concentrated in vacuo and an aqueous 0.1 HCl solution was added under precipitation of a green solid. The solid was collected by filtration, washed with cold 0.1 N HCl and dried in the oven. The product was obtained as green solid in 95% yield (135 mg, 0.53 mmol). ¹H NMR (400 MHz, DMSO) δ 13.66 (s, 1H), 10.55 (s, 1H), 8.05 (s, 1H), 7.70 (dd, J=7.8, 1.1 Hz, 1H), 7.58 (dd, J=7.8, 1.0 Hz, 1H), 7.42-7.36 (m, 1H), 7.27 (d, J=7.1 Hz, 1H), 7.09-7.02 (m, 2H), 6.93 (t, J=7.4 Hz, 1H). ¹³C NMR (101 MHz, DMSO) δ 169.2, 167.6, 150.1, 140.2, 133.9, 132.1, 132.1, 125.5, 124.6, 123.9, 122.1, 121.1, 119.2, 119.1. TLC-MS (ESI−): calcd. m/z 254.07 for C₁₄H₁₀N₂O₃, found 252.7 [M−H]⁻.

### Preparation of 2, 3 and 4

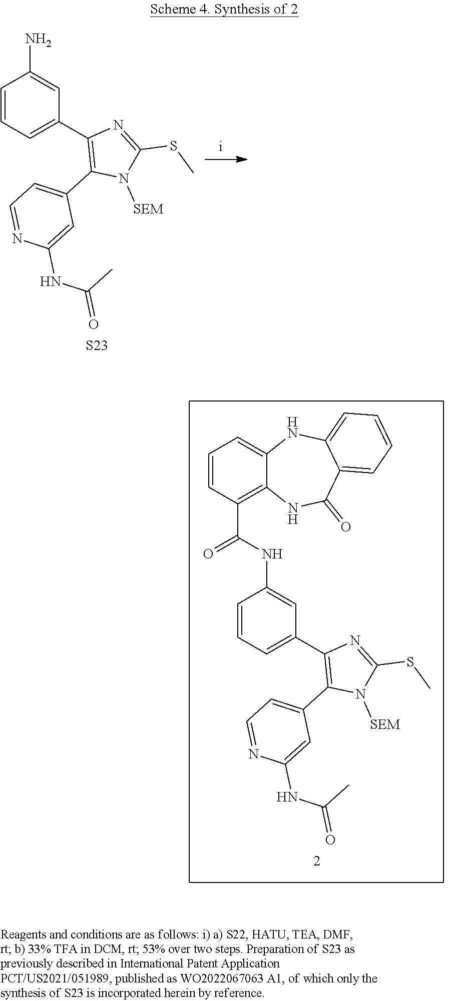

N-(3-(5-(2-Acetamidopyridin-4-yl)-2-(methylthio)-1H-imidazol-4-yl)phenyl)-11-oxo-10,11-dihydro-5H-dibenzo[b,e][1,4]diazepine-9-carboxamide (2). To begin, 50 mg (0.11 mmol) of S23, 35 mg (0.14 mmol) of S22, and 61 mg (0.16 mmol) of HATU were dissolved in 2 mL of DMF, and to the mixture was added 45 μL (0.32 mmol) of triethylamine. The reaction mixture was stirred overnight at ambient temperature. Brine was added to reaction mixture, and the aqueous layer was extracted several times with EtOAc. The combined organic layers were dried over Na₂SO₄, and solvents were removed in vacuo. The residue was purified via flash chromatography (SiO₂; Hex/EtOAc/MeOH 20:75:5) (identification of the intermediate via TLC-MS (ESI+): calcd. m/z 705.26 for C₃₇H₃₉N₇O₄SSi, found 728.3 [M+Na]⁺) and then directly dissolved in a 33% TFA/DCM mixture. After stirring overnight at room temperature, solvents were evaporated and to the residue was added a saturated aqueous NaHCO₃ solution, and the aqueous layer was extracted several times with EtOAc. Combined organic layers were dried over Na₂SO₄, and solvents were removed in vacuo. The residue was purified via flash chromatography (SiO₂; EtOAc/(10% IPA in EtOAc) 20:80) to obtain a 53% yield (40 mg, 0.07 mmol) of a white solid. As mixture of tautomers: ¹H NMR (400 MHz, DMSO) δ 12.84-12.69 (m, 1H), 10.63-10.50 (m, 1H), 10.49-10.31 (m, 1H), 10.11-9.98 (m, 1H), 8.39-8.11 (m, 2H), 8.10-8.02 (m, 1H), 7.97-7.86 (m, 1H), 7.82-7.70 (m, 1H), 7.70-7.63 (m, 1H), 7.48-7.40 (m, 1H), 7.39-7.27 (m, 2H), 7.26-7.21 (m, 1H), 7.19 (d, J=7.6 Hz, 1H), 7.16-7.11 (m, 1H), 7.11-7.03 (m, 2H), 6.94 (t, J=7.5 Hz, 1H), 2.63 (s, 3H), 2.12-2.00 (m, 3H). ¹³C NMR (101 MHz, DMSO) δ 169.2, 169.0, 167.7, 167.0, 166.8, 152.6, 152.4, 150.2, 150.2, 148.2, 147.6, 143.7, 143.4, 142.3, 141.3, 139.6, 139.4, 139.1, 138.8, 134.8, 134.3, 133.6, 133.6, 132.2, 132.1, 131.0, 130.6, 129.2, 129.0, 128.5, 126.1, 125.8, 125.7, 124.3, 124.2, 124.2, 123.4, 123.3, 123.3, 122.7, 122.5, 122.5, 121.2, 120.3, 120.2, 119.2, 117.2, 116.5, 110.9, 110.5, 23.6, 15.1, 15.0. HRMS (ESI): exact mass calcd, for C₃₁H₂₅N₇O₃S [M+Na]⁺: 598.16318, found: 598.16425.

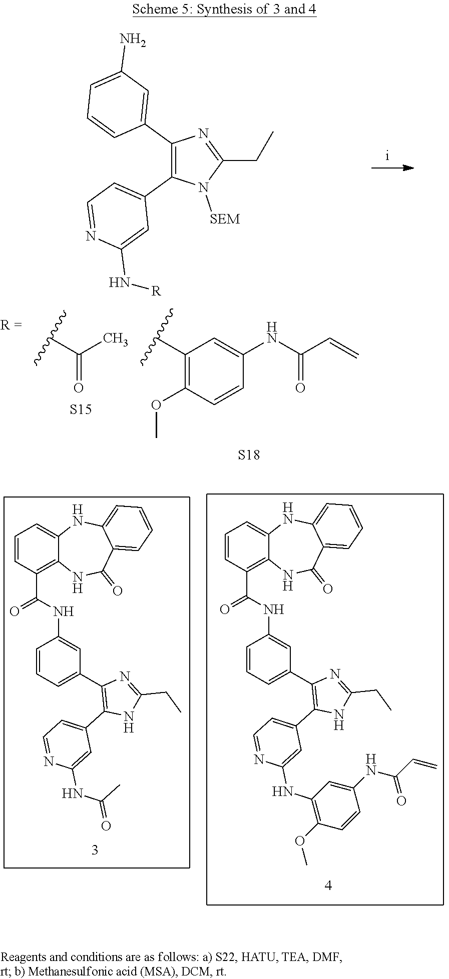

### N-(3-(5-(2-Acetamidopyridin-4-yl)-2-ethyl-1H-imidazol-4-yl)phenyl)-11-oxo-10,11-dihydro-5H-dibenzo[b,e][1,4]diazepine-9-carboxamide (3)

To begin, 50 mg (0.11 mmol) of S15, 31 mg (0.12 mmol) of S22, and 55 mg (0.14 mmol) of HATU were dissolved in 2 mL of DMF, and to the mixture was added 31 μL (0.22 mmol) of triethylamine. The reaction mixture was stirred overnight at ambient temperature. Demineralized water was added to reaction mixture under precipitation of a yellowish solid, which was collected by filtration and dried in the oven. (Identification of the intermediate via TLC-MS (ESI+): calcd. m/z 687.30 for C₃₈H₄₁N₇O₄Si, found 710.8 [M+Na]⁺) The solid was then directly dissolved 2 ml of DCM and the mixture was cooled down to 0° C. After addition of 100 μL MSA, the mixture was slowly warmed up to room temperature and stirred for 1 h. To the mixture was added a saturated aqueous NaHCO₃ solution, and the aqueous layer was extracted several times with EtOAc. Combined organic layers were dried over Na₂SO₄, and solvents were removed in vacuo. The residue was purified via flash chromatography (SiO₂; DCM/MeOH 90:10) to obtain a 50% yield (31 mg, 0.06 mmol) of a white solid. As mixture of tautomers: ¹H NMR (400 MHz, DMSO) δ 12.41-12.19 (m, 1H), 10.63-10.48 (m, 1H), 10.48-10.26 (m, 1H), 10.16-9.99 (m, 1H), 8.44-8.05 (m, 3H), 8.00-7.86 (m, 1H), 7.82-7.62 (m, 2H), 7.49-7.28 (m, 3H), 7.24 (d, J=7.8 Hz, 1H), 7.19 (d, J=7.7 Hz, 1H), 7.16-7.00 (m, 3H), 6.94 (t, J=7.5 Hz, 1H), 2.77-2.64 (m, 2H), 2.04 (s, 3H), 1.28 (t, J=7.6 Hz, 3H). ¹³C NMR (101 MHz, DMSO) δ 168.9, 167.7, 167.0, 152.4, 150.2, 149.9, 147.4, 144.5, 141.2, 139.1, 133.6, 132.7, 132.1, 131.4, 129.1, 129.0, 128.7, 125.7, 124.2, 123.3, 122.7, 122.5, 121.2, 120.3, 119.9, 119.2, 116.6, 110.5, 23.9, 21.2, 12.7a. HRMS (ESI): exact mass calcd, for C₃₁H₂₅N₇O₃S [M+Na]⁺: 580.20676, found: 580.20779.

### N-(3-(5-(2-((5-Acrylamido-2-methoxyphenyl)amino)pyridin-4-yl)-2-ethyl-1H-imidazol-4-yl)phenyl)-11-oxo-10,11-dihydro-5H-dibenzo[b,e][1,4]diazepine-9-carboxamide (4)

To begin, 50 mg (0.09 mmol) of S18, 26 mg (0.10 mmol) of S22, and 49 mg (0.13 mmol) of HATU were dissolved in 2 mL of DMF, and to the mixture was added 36 μL (0.26 mmol) of triethylamine. The reaction mixture was stirred overnight at ambient temperature. Demineralized water was added to reaction mixture under precipitation of a yellowish solid, which was collected by filtration and dried in the oven. (Identification of the intermediate via TLC-MS (ESI+): calcd. m/z 820.35 for C₄₆H₄₈N₈O₅Si, found 844.0 [M+Na]⁺) The solid was then directly dissolved 2 ml of DCM and the mixture was cooled down to 0° C. After addition of 100 μL MSA, the mixture was slowly warmed up to room temperature and stirred for 1 h. To the mixture was added a saturated aqueous NaHCO₃ solution, and the aqueous layer was extracted several times with EtOAc. Combined organic layers were dried over Na₂SO₄, and solvents were removed in vacuo. The residue was purified via flash chromatography (SiO₂; n-hexane/EtOAc 15:85 to EtOAc/IPA 85:15) to obtain a 30% yield (18 mg, 0.03 mmol) of a white solid. As mixture of tautomers: ¹H NMR (400 MHz, DMSO) δ 12.25-12.16 (m, 1H), 10.65-10.53 (m, 1H), 10.11-10.03 (m, 1H), 9.98-9.90 (m, 1H), 8.36-8.29 (m, 1H), 8.10-8.06 (m, 1H), 8.04-7.99 (m, 1H), 7.98-7.93 (m, 1H), 7.88-7.69 (m, 2H), 7.67 (d, J=7.8 Hz, 1H), 7.46-7.42 (m, 1H), 7.42-7.40 (m, 1H), 7.39-7.35 (m, 1H), 7.34-7.32 (m, 1H), 7.30-7.20 (m, 2H), 7.19-7.09 (m, 2H), 7.05 (d, J=8.0 Hz, 1H), 6.96-6.89 (m, 2H), 6.73 (dd, J=5.3, 0.8 Hz, 1H), 6.48-6.40 (m, 1H), 6.24-6.16 (m, 1H), 5.71-5.65 (m, 1H), 3.82-3.75 (m, 3H), 2.74-2.67 (m, 2H), 1.29 (t, J=7.6 Hz, 3H). HRMS (ESI): exact mass calcd, for C₄₀H₃₄N₈O₄ [M+Na]⁺: 713.25952, found: 713.25995.

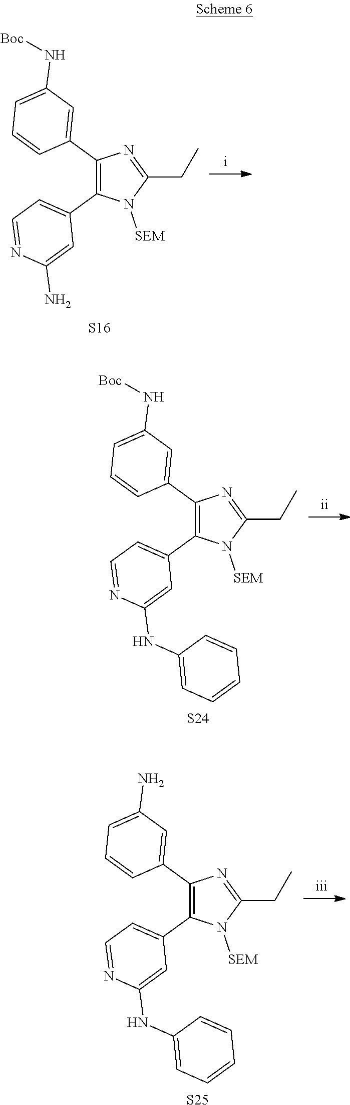 [SMILES: CCc1nc(-c2cccc(NC(=O)OC(C)(C)C)c2)c(-c2ccnc(N)c2)n1COCC[Si](C)(C)C ; "S16" ; CCc1nc(-c2cccc(NC(=O)OC(C)(C)C)c2)c(-c2ccnc(Nc3ccccc3)c2)n1COCC[Si](C)(C)C ; "S24" ; CCc1nc(-c2cccc(N)c2)c(-c2ccnc(Nc3ccccc3)c2)n1COCC[Si](C)(C)C ; "S25"]

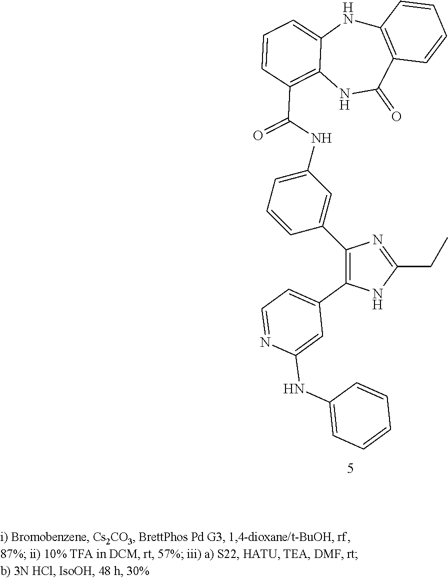 [SMILES: CCc1nc(-c2cccc(NC(=O)c3cccc4c3NC(=O)c3ccccc3N4)c2)c(-c2ccnc(Nc3ccccc3)c2)[nH]1]

tert-butyl (3-(2-ethyl-5-(2-(phenylamino)pyridin-4-yl)-1-((2-(trimethylsilyl)ethoxy)methyl)-1H-imidazol-4-yl)phenyl)carbamate (S24). To begin, 350 mg (0.69 mmol) of S16, 162 mg (1.03 mmol) of bromobenzene, and 336 mg of Cs₂CO₃ (1.03 mmol) in 7 mL of a mixture of 1,4-dioxane/t-BuOH (4+1) was degassed three times by evacuating and backfilling with argon under stirring. The mixture was added 31 mg of BrettPhos Pd G3 (5 mol %), and another three cycles of evacuation and argon backfilling were carried out. The solution was stirred under reflux for 5 h. After cooling to ambient temperature, Celite was added to the mixture, and solvents were removed in vacuo. Purification via flash chromatography (SiO₂; n-hexane/EtOAc 40:60) yielded 87% (350 mg, 0.60 mmol) of a yellow solid. ¹H NMR (400 MHz, DMSO) δ 9.28 (s, 1H), 9.06 (s, 1H), 8.23 (d, J=5.2 Hz, 1H), 7.75-7.72 (m, 1H), 7.61-7.59 (m, 1H), 7.59-7.57 (m, 1H), 7.29-7.25 (m, 1H), 7.24-7.20 (m, 2H), 7.10 (t, J=7.9 Hz, 1H), 6.93-6.90 (m, 1H), 6.89-6.85 (m, 1H), 6.74 (s, 1H), 6.73-6.70 (m, 1H), 5.14 (s, 2H), 3.35-3.31 (m, 3H), 2.80 (q, J=7.5 Hz, 2H), 1.43 (s, 9H), 1.32 (t, J=7.5 Hz, 3H), 0.78-0.73 (m, 2H), −0.10 (s, 9H). ¹³C NMR (101 MHz, DMSO) δ 156.32, 152.73, 150.37, 148.03, 141.44, 139.52, 139.40, 136.29, 134.79, 128.55, 128.22, 126.38, 120.64, 120.59, 118.14, 117.02, 116.48, 115.52, 111.87, 78.84, 71.74, 65.10, 28.09, 19.76, 17.19, 12.12, −1.50. TLC-MS (ESI+): calcd.m/z 585.31 for C₃₃H₄₃N₅O₃Si, found 608.3 [M+Na]⁺.

4-(4-(3-Aminophenyl)-2-ethyl-1-((2-(trimethylsilyl)ethoxy)methyl)-1H-imidazol-5-yl)-N-phenylpyridin-2-amine (S25). 320 mg (0.55 mmol) of S24 was dissolved in 18 ml of a mixture of TFA/DCM 10% v/v. The solution was stirred for 24 h at ambient temperature. The reaction mixture was concentrated in vacuo and the residue was quenched with a saturated aqueous NaHCO₃ solution, whereupon the aqueous layer was extracted several times with EtOAc. The combined organic layers were dried over Na₂SO₄, filtered, and the solvents removed in vacuo. The crude product was purified via flash chromatography (SiO₂; n-hexane/EtOAc/MeOH 30:65:5) to give the product as a light yellow solid in a 57% yield (150 mg, 0.31 mmol). ¹H NMR (400 MHz, DMSO) δ 9.07 (s, 1H), 8.23 (d, J=5.1 Hz, 1H), 7.60 (s, 1H), 7.58 (s, 1H), 7.25-7.20 (m, 2H), 6.90-6.87 (m, 2H), 6.86-6.82 (m, 1H), 6.76 (s, 1H), 6.74-6.70 (m, 1H), 6.45-6.41 (m, 1H), 6.41-6.36 (m, 1H), 5.12 (s, 2H), 4.99 (s, 2H), 3.35-3.30 (m, 3H), 2.78 (q, J=7.5 Hz, 2H), 1.31 (t, J=7.5 Hz, 3H), 0.78-0.72 (m, 2H), −0.10 (s, 9H). ¹³C NMR (101 MHz, DMSO) δ 156.2, 150.1, 148.6, 147.9, 141.4, 139.8, 137.0, 134.9, 128.6, 128.4, 125.9, 120.6, 118.1, 115.6, 114.7, 112.7, 112.3, 111.9, 71.7, 65.1, 19.8, 17.2, 12.1, −1.5. TLC-MS (ESI+): calcd.m/z 485.26 for C₂₈H₃₅N₅OSi, found 508.2 [M+Na]⁺.

N-(3-(2-ethyl-5-(2-(phenylamino)pyridin-4-yl)-1H-imidazol-4-yl)phenyl)-11-oxo-10,11-dihydro-5H-dibenzo[b,e][1,4]diazepine-9-carboxamide (5). To begin, 50 mg (0.10 mmol) of S25, 30 mg (0.12 mmol) of S22, and 59 mg (0.15 mmol) of HATU were dissolved in 1 mL of DMF, and to the mixture was added 29 μL (0.21 mmol) of triethylamine. The reaction mixture was stirred overnight at ambient temperature. Demineralized water was added to the reaction mixture under precipitation of a yellowish solid, which was collected by filtration and dried in the oven. (Identification of the intermediate via TLC-MS (ESI+): calcd. m/z 721.32 for C₄₂H₄₃N₇O₃Si, found 744.8 [M+Na]⁺) The solid was then directly dissolved 2 ml of 3 N HCl in IsoOH and the mixture stirred for 48 h. To the mixture was added a saturated aqueous NaHCO₃ solution, and the aqueous layer was extracted several times with EtOAc. Combined organic layers were dried over Na₂SO₄, and solvents were removed in vacuo. The residue was purified via flash chromatography (SiO₂; DCM/MeOH 90:10) to obtain a 30% yield (18 mg, 0.03 mmol) of a white solid. ¹H NMR (400 MHz, DMSO) δ 12.24 (s, 1H), 10.65 (s, 1H), 10.05 (s, 1H), 8.95 (s, 1H), 8.16-7.79 (m, 4H), 7.68 (d, J=7.7 Hz, 1H), 7.60-7.52 (m, 2H), 7.50-7.30 (m, 3H), 7.27-7.08 (m, 6H), 7.05 (d, J=7.9 Hz, 1H), 6.93 (t, J=7.4 Hz, 1H), 6.86-6.75 (m, 2H), 2.75-2.67 (m, 2H), 1.29 (t, J=7.4 Hz, 3H). TLC-MS (ESI+): calcd.m/z 591.24 for C₃₆H₂₉N₇O₂, found 592.6 [M+H]+.

Although the present disclosure has been described with respect to one or more particular embodiments and/or examples, it will be understood that other embodiments and/or examples of the present disclosure may be made without departing from the scope of the present disclosure.

1. A compound having the following structure 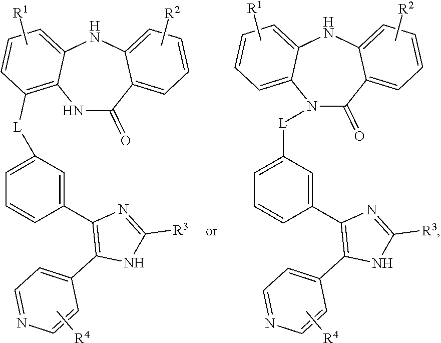
wherein
R¹ is a halogen, a substituted aliphatic group, an unsubstituted aliphatic group, or is absent;
R² is a halogen, a substituted aliphatic group, an unsubstituted aliphatic group, or is absent;
R³ is a substituted aliphatic group; an unsubstituted aliphatic group; a thioether group, or absent;
R⁴ is an amide or a secondary amine; and
L is a linker.

2. The compound according to claim 1, wherein
R¹ is —I, —F, —Br, —Cl, a substituted or unsubstituted alkyl group, or absent;
R² is —I, —F, —Br, —Cl, a substituted or unsubstituted alkyl group, or absent;
R³ is 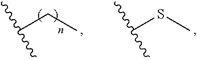
or absent, wherein n is 0, 1, 2, or 3;
R⁴ is 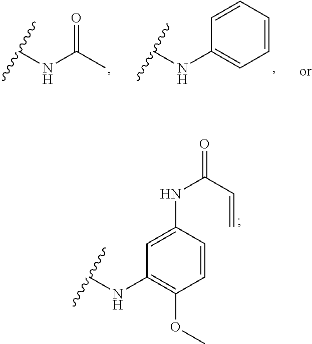
and/or 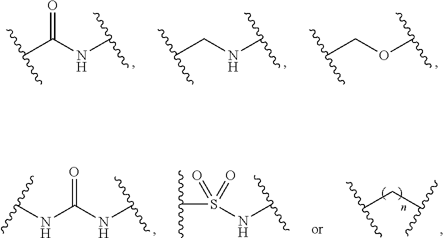
L is
wherein n is 1, 2, or 3.

3. The compound according to claim 1, wherein the compound has the following structure:

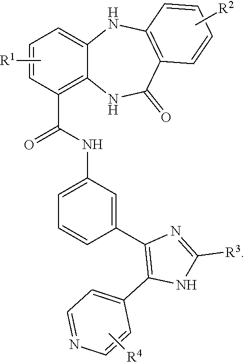

4. The compound according to claim 1, wherein the compound has the following structure:

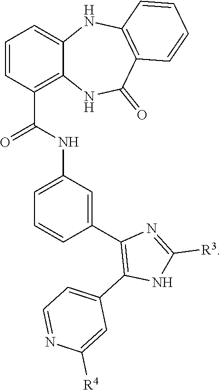

5. The compound according to claim 1, wherein the compound has the following structure: 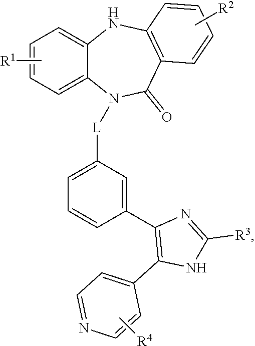
wherein L is 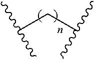
and n is 1, 2, or 3.

6. The compound according to claim 1, wherein the compound has the following structure:

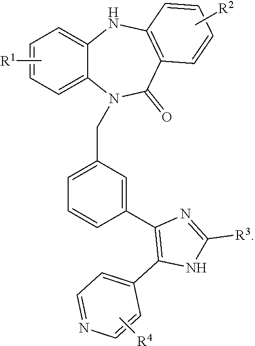

7. The compound according to claim 1, wherein the compound has the following structure: 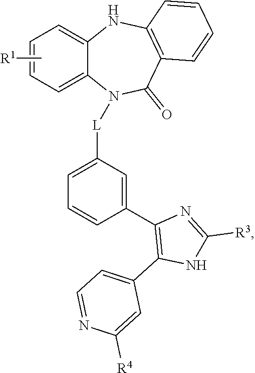
wherein L is 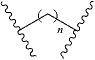
and n is 1, 2, or 3.

8. The compound according to claim 1, wherein the compound has the following structure:

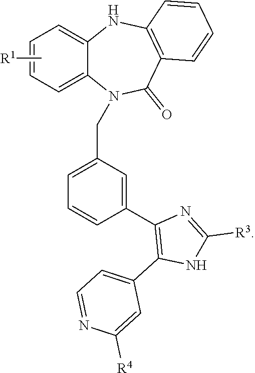

9. The compound according to claim 1, wherein the compound has the following structure:

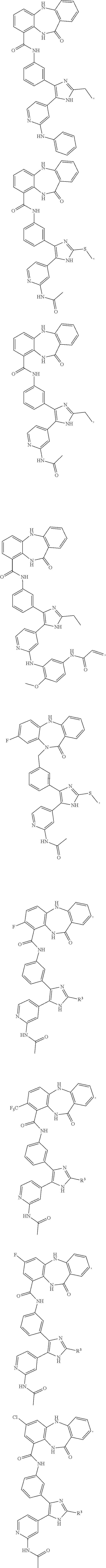

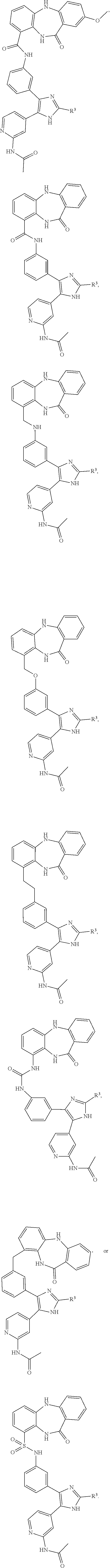

10. A composition comprising the compound according to claim 1.

11. The composition according to claim 10, further comprising a pharmaceutically acceptable carrier.

12. A method of treating an individual having or suspected of having cancer, comprising:
administering a therapeutically effective amount of the compound according to claim 1 or a composition comprising one or more compounds according to claim 1 to the individual.

13. The method according to claim 12, wherein the individual is a human or non-human animal.

14. The method according to claim 12, wherein the composition inhibits a mutant epidermal growth factor receptor (EGFR).

15. The method according to claim 12, wherein the cancer is lung cancer or breast cancer.

16. The method according to claim 15, wherein the lung cancer is an EGFR non-small cell lung cancer.

17. The method according to claim 16, wherein the non-small cell lung cancer has one or more of the following mutations: L858R, exon19del (e.g., delE756-A750, delL747-A750insP, delL747-T751), T790M, and/or C797S.

18. The method according to claim 17, wherein the exon19del mutation is delE756-A750, delL747-A750insP, or delL747-T751.

19. The method according to claim 15, wherein the breast cancer is HER2 over-expressing breast cancer.

20. A kit, comprising:
a composition comprising the compound according to claim 1 or constituents to prepare a composition comprising the compound according to claim 1.
